(19) United States
(12) Patent Application Publication
(10) Pub. No.: US 2026/0234263 A1
(43) Pub. Date: Aug. 13, 2026

(54) HUMAN ANTI-PD-L2 ANTIBODY AND ITS USES THEREOF

(71) Applicant: MABTREE BIOLOGICS AG, Basel (CH)

(72) Inventors: Ratna Monica Alexander, Basel (CH); Andhuvan Raj Eruthayaraj, Basel (CH); Rajesh Jayachandran, Basel (CH); Chandni Sood, Basel (CH)

(73) Assignee: MABTREE BIOLOGICS AG, Basel (CH)

(21) Appl. No.: 19/153,498

(22) PCT Filed: Feb. 3, 2024
(86) PCT No.: PCT/IB2024/050995

§ 371 Date: Aug. 4, 2025

(30) Foreign Application Priority Data
Feb. 4, 2023 (IN) 202341007181

(51) Int. Cl. C07K 16/28; A61K 39/00; A61K 39/395; A61P 35/00

(52) U.S. Cl. CPC C07K 16/2827; A61K 39/3955; A61P 35/00; A61K 2039/505; C07K 2317/24; C07K 2317/565; C07K 2317/76; C07K 2317/92

(57) ABSTRACT

The present invention provides antibodies or antigen-binding fragments thereof which specifically bind to human PD-L2, and novel complementarity determining regions CDR1, CDR2, and CDR3 for each heavy chain and light chain of the antibodies. The antibodies or antigen-binding fragments have application in enhancing T-cell function, and treatment of cancer and infectious diseases, acute or chronic.

### RELATED APPLICATIONS

This application claims priority from the PCT application No. PCT/IB2024/050995 filed on 3 Feb. 2024, which claims priority to the Indian patent application numbered 202341007181 titled “HUMAN ANTI-PD-L2 ANTIBODY AND ITS USES THEREOF” filed on 4 Feb. 2023. Both applications are incorporated in full herein by reference

### FIELD OF INVENTION

The present invention relates to antibody that binds to Programmed Death Ligand 2 (PD-L2), also called as cluster of differentiation 273 (CD273). Further, the invention relates to antibody or an antibody fragment that binds to PD-L2 for its application to enhance T-cell function including the treatment of cancer and acute/chronic infections.

### BACKGROUND OF THE INVENTION

The programmed death-1 (PD-1, CD279) receptor has two ligands programmed death ligand 1 (PD-L1, CD274, B7-H1), and programmed death ligand 2 (PD-L2, CD273, B7-DC). Interaction of PD-1 with its ligands is one of the key players of immune checkpoint pathways inhibiting T-Cell activation. Though the role of PD-L1, and PD-L2 in tumour microenvironment is still debateable, there are several studies which indicate the role of both ligands in several tumour types. Cancer cells evade immune response via exploiting various mechanisms including exploitation of PD-1/PD-L1 and PD-1/PD-L2 interaction which suppress T-cell activation leading to T cell immunosuppression, termed anergy or T-cell exhaustion, and as well attenuates cytotoxic secretion thereby favouring cancer to grow by mitigating anti-tumour immune responses. Antibodies against PD-1, PD-L1, and PD-L2 compete for the ligand receptor binding and inhibit this interaction, thus, enhancing T-cell activation.

Although PD-L2 has been historically considered a minor ligand, it binds to PD-1 with a two- to six-fold higher affinity as compared to PD-L1. PD-L2 can be expressed by immune, stromal, or tumour cells. Hitherto, most of the focus has been on targeting the PD-1/PD-L1 interaction for immunotherapy and several monoclonal antibodies specifically designed to inhibit the PD-1/PD-L1 axis (e.g., nivolumab, pembrolizumab, atezolizumab, durvalumab, etc.) are now available for the treatment of solid and hematological malignancies.

Few antibodies targeting PD-1/PD-L2 interaction have been developed for immunotherapy against cancer/tumour.

WO2010036959A2 describes novel composite, human monoclonal antibodies which specifically bind to human PD-I, human PD-L1, and human PD-L2. Though the human composite antibodies against PD-1, and PD-L1 showed better IC50 values than the mouse variants, the human composite antibody against PD-L2 showed lower IC50 value than its mouse variant, and is as high as 0.5 μg/ml.

WO2017/053250A1 discloses anti-human PD-L2 antibodies provides antibodies for immunohistochemical detection of human PD-L2 expression in tissue sample. However, its use in therapy was not shown.

US20200369772A1 discloses anti-human PD-L2 antibodies for cancer therapy. The EC50 values of provided antibodies are significantly less than the standard reference antibody 24F.10C12, and their binding efficiencies have been shown in various in vitro using mammalian cancer culture cells. However, their efficiency in cancer therapy has not been shown.

Taking into consideration the drawbacks of the prior art the present invention provides antibodies against PD-L2 to meet the medical needs.

### OBJECT(S) OF THE INVENTION

The main object of the present invention is to provide novel human antibodies or antigen-binding fragments against PD-L2 for use in enhancing T-cell function and having an application for the treatment of cancer and infectious diseases, acute or chronic.

Another object of the present invention is to provide a nucleic acid encoding the antibodies or an antigen-binding fragments thereof against PD-L2.

Yet another object of the present invention to provide a composition for preventing or treating cancer, containing the antibodies or an antigen-binding fragments thereof.

Yet another object of the present invention to provide a composition for preventing or treating infectious diseases, containing the antibodies or an antigen-binding fragments thereof.

### SUMMARY OF THE INVENTION

The present invention relates to human anti-PD-L2 antibodies or the antigen binding fragments thereof, which specifically bind to human PD-L2 such that PD-L2 binding to PD-1 is blocked.

The present invention relates to an antibodies or antigen-binding fragments thereof which specifically bind to human PD-L2 comprising a light chain, and a heavy chain. Further, the invention provides light chain comprising light chain variable region with complementarity determining regions LCDR1, LCDR2, and LCDR3 sequences. The invention also provides heavy chain comprising heavy chain variable region with complementarity determining regions HCDR1, HCDR2, and HCDR3 sequences.

In one of the embodiments, the present invention provides an antibody or antigen-binding fragment which specifically binds to human PD-L2 comprising a light chain variable region selected from the group comprising of Seq. ID 1, Seq. ID 2, Seq. ID 3, Seq. ID 7, Seq. ID 8, Seq. ID 9, Seq. ID 13, Seq. ID 14, Seq. ID 15, Seq. ID 19, Seq. ID 20, Seq. ID 21, Seq. ID 25, Seq. ID 26, Seq. ID 27, Seq. ID 31, Seq. ID 32, Seq. ID 33, Seq. ID 37, Seq. ID 38, Seq. ID 39, Seq. ID 43, Seq. ID 44, and Seq. ID 45.

In one of the embodiments, the present invention provides an antibody or antigen-binding fragment which specifically binds to human PD-L2 comprising a light chain variable region LCDR1 selected from the group comprising of Seq. ID 1, Seq. ID 7, Seq. ID 13, Seq. ID 19, Seq. ID 25, Seq. ID 31, Seq. ID 37, and Seq. ID 43.

In one of the embodiments, the present invention provides an antibody or antigen-binding fragment which specifically binds to human PD-L2 comprising a light chain variable region LCDR2 selected from the group comprising of Seq. ID 2, Seq. ID 8, Seq. ID 14, Seq. ID 20, Seq. ID 26, Seq. ID 32, Seq. ID 38, and Seq. ID 44.

In one of the embodiments, the present invention provides an antibody or antigen-binding fragment which specifically binds to human PD-L2 comprising a light chain variable region LCDR3 selected from the group comprising of Seq. ID 3, Seq. ID 9, Seq. ID 15, Seq. ID 21, Seq. ID 27, Seq. ID 33, Seq. ID 39, and Seq. ID 45.

In one of the embodiments, the present invention provides light chain variable region of an antibody or antigen-binding fragment which specifically binds to human PD-L2 comprising LCDR1, LCDR2, and LCDR3, respectively, set forth in Seq. ID Nos. 1, 2, and 3; Seq. ID Nos. 7, 8, and 9; Seq. ID Nos. 13, 14, and 15; Seq. ID Nos. 19, 20, and 21; Seq. ID Nos. 25, 26, and 27; Seq. ID Nos. 31, 32, and 33; Seq. ID Nos. 37, 38, and 39; or Seq. ID Nos. 43, 44, and 45.

In one of the embodiments, the present invention provides an antibody or antigen-binding fragment which specifically binds to human PD-L2 comprising a heavy chain variable region selected from the group comprising of Seq. ID 4, Seq. ID 5, Seq. ID 6, Seq. ID 10, Seq. ID 11, Seq. ID 12, Seq. ID 16, Seq. ID 17, Seq. ID 18, Seq. ID 22, Seq. ID 23, Seq. ID 24, Seq. ID 28, Seq. ID 29, Seq. ID 30, Seq. ID 34, Seq. ID 35, Seq. ID 36, Seq. ID 40, Seq. ID 41, Seq. ID 42, Seq. ID 46, Seq. ID 47, and Seq. ID 48.

In one of the embodiments, the present invention provides an antibody or antigen-binding fragment which specifically binds to human PD-L2 comprising a heavy chain variable region HCDR1 selected from the group comprising of Seq. ID 4, Seq. ID 10, Seq. ID 16, Seq. ID 22, Seq. ID 28, Seq. ID 34, Seq. ID 40, and Seq. ID 46.

In one of the embodiments, the present invention provides an antibody or antigen-binding fragment which specifically binds to human PD-L2 comprising a heavy chain variable region HCDR2 selected from the group comprising of Seq. ID 5, Seq. ID 11, Seq. ID 17, Seq. ID 23, Seq. ID 29, Seq. ID 35, Seq. ID 41, and Seq. ID 47.

In one of the embodiments, the present invention provides an antibody or antigen-binding fragment which specifically binds to human PD-L2 comprising a heavy chain variable region HCDR3 selected from the group comprising of Seq. ID 6, Seq. ID 12, Seq. ID 18, Seq. ID 24, Seq. ID 30, Seq. ID 36, Seq. ID 42, and Seq. ID 48.

In one of the embodiments, the present invention provides heavy chain variable region of an antibody or antigen-binding fragment which specifically binds to human PD-L2 comprising HCDR1, HCDR2, and HCDR3, respectively, set forth in Seq. ID Nos. 4, 5, and 6; Seq. ID Nos. 10, 11, and 12; Seq. ID Nos. 16, 17, and 18; Seq. ID Nos. 22, 23, and 24; Seq. ID Nos. 28, 29, and 30; Seq. ID Nos. 34, 35, and 36; Seq. ID Nos. 40, 41, and 42; or Seq. ID Nos. 46, 47, and 48.

In one of the embodiments, the present invention provides an antibody or antigen-binding fragment which specifically binds to human PD-L2 comprising LCDR1, LCDR2, and LCDR3, respectively, set forth in Seq. ID Nos. 1, 2, and 3; and HCDR1, HCDR2, and HCDR3, respectively, set forth in Seq. ID Nos. 4, 5, and 6.

In one of the embodiments, the present invention provides an antibody or antigen-binding fragment which specifically binds to human PD-L2 comprising LCDR1, LCDR2, and LCDR3, respectively, set forth in Seq. ID Nos. 7, 8, and 9; and HCDR1, HCDR2, and HCDR3, respectively, set forth in Seq. ID Nos. 10, 11 and 12.

In one of the embodiments, the present invention provides an antibody or antigen-binding fragment which specifically binds to human PD-L2 comprising LCDR1, LCDR2, and LCDR3, respectively, set forth in Seq. ID Nos. 13, 14, and 15; and HCDR1, HCDR2, and HCDR3, respectively, set forth in Seq. ID Nos. 16, 17, and 18.

In one of the embodiments, the present invention provides an antibody or antigen-binding fragment which specifically binds to human PD-L2 comprising LCDR1, LCDR2, and LCDR3, respectively, set forth in Seq. ID Nos. 19, 20, and 21; and HCDR1, HCDR2, and HCDR3, respectively, set forth in Seq. ID Nos. 22, 23, and 24.

In one of the embodiments, the present invention provides an antibody or antigen-binding fragment which specifically binds to human PD-L2 comprising LCDR1, LCDR2, and LCDR3, respectively, set forth in Seq. ID Nos. 25, 26, and 27; and HCDR1, HCDR2, and HCDR3, respectively, set forth in Seq. ID Nos. 28, 29, and 30.

In one of the embodiments, the present invention provides an antibody or antigen-binding fragment which specifically binds to human PD-L2 comprising LCDR1, LCDR2, and LCDR3, respectively, set forth in Seq. ID Nos. 31, 32, and 33; and HCDR1, HCDR2, and HCDR3, respectively, set forth in Seq. ID Nos. 34, 35, and 36.

In one of the embodiments, the present invention provides an antibody or antigen-binding fragment which specifically binds to human PD-L2 comprising LCDR1, LCDR2, and LCDR3, respectively, set forth in Seq. ID Nos. 37, 38, and 39; and HCDR1, HCDR2, and HCDR3, respectively, set forth in Seq. ID Nos. 40, 41 and 42.

In one of the embodiments, the present invention provides an antibody or antigen-binding fragment which specifically binds to human PD-L2 comprising LCDR1, LCDR2, and LCDR3, respectively, set forth in Seq. ID Nos. 43, 44, and 45; and HCDR1, HCDR2, and HCDR3, respectively, set forth in Seq. ID Nos. 46, 47, and 48.

In one of the embodiments, the present invention provides an antibody or antigen-binding fragment which specifically binds to human PD-L2 comprising a light chain selected from the group comprising Seq. ID 49, Seq. ID 50, Seq. ID 51, Seq. ID 52, Seq. ID 53, Seq. ID 54, Seq. ID 55, and Seq. ID 56.

In one of the embodiments, the present invention provides an antibody or antigen-binding fragment which specifically binds to human PD-L2 comprising a light chain selected from the group comprising Seq. ID 50, Seq. ID 51, Seq. ID 54, and Seq. ID 56 or sequences with 85-100%, wherein, the light chain variable region of said light chain sequences comprise amino acid M, or L in position 4, amino acid T or A in position 10, amino acid V or L in position 13, amino acid G or A in position 68, amino acid E or D in position 70, amino acid Q or E in position 79, amino acid K or R in position 103, and amino acid V or L in position 105.

In one of the embodiments, the present invention provides an antibody or antigen-binding fragment which specifically binds to human PD-L2 comprising a light chain selected from the group comprising Seq. ID 49, and Seq. ID 52, or sequences with 85-100% wherein, the light chain variable region of said light chain sequences comprise amino acid T, or K in position 17, amino acid V or I in position 59, amino acid S or T in position 73, amino acid G or A in position 75, amino acid S or T in position 77, amino acid S or T in position 81, and amino acid E or G in position 82.

In one of the embodiments, the present invention provides an antibody or antigen-binding fragment which specifically binds to human PD-L2 comprising a light chain selected from the group comprising Seq. ID 53, and Seq. ID 55, or sequences with 85-100% wherein, the light chain variable region of said light chain sequences comprise amino acid D, or N in position 1, amino acid M or L in position 4, amino acid I or T in position 5, amino acid V or L in position 11, amino acid S or G in position 65, amino acid S or Y in position 67, amino acid D or H in position 70, and amino acid S or G in position 77.

In one of the embodiments, the present invention provides an antibody or antigen-binding fragment which specifically binds to human PD-L2 comprising a heavy chain selected from the group comprising of Seq. ID 57, Seq. ID 58, Seq. ID 59, Seq. ID 60, Seq. ID 61, Seq. ID 62, Seq. ID 63, and Seq. ID 64. Further, the invention provides heavy chain variable region of said heavy chain sequences, wherein, amino acid in position 1 is Q or E, amino acid in position 5 is V or Q, amino acid in position 74 is K or E, amino acid in position 114, 115, 116 or 118 is T, M, or L.

In one of the embodiments, the present invention provides nucleic acid sequences encoding light chain of an antibody or antigen-binding fragment which specifically binds to human PD-L2 selected from the group comprising of Seq. ID 65, Seq. ID 66, Seq. ID 67, Seq. ID 68, Seq. ID 69, Seq. ID 70, Seq. ID 71, and Seq. ID 72, encoding light chain polypeptide sequences Seq. ID 49, Seq. ID 50, Seq. ID 51, Seq. ID 52, Seq. ID 53, Seq. ID 54, Seq. ID 55, and Seq. ID 56 respectively.

In one of the embodiments, the present invention provides nucleic acid sequences encoding heavy chain of an antibody or antigen-binding fragment which specifically binds to human PD-L2 selected from the group comprising of Seq. ID 73, Seq. ID 74, Seq. ID 75, Seq. ID 76, Seq. ID 77, Seq. ID 78, Seq. ID 79, and Seq. ID 80, encoding light chain polypeptide sequences Seq. ID 57, Seq. ID 58, Seq. ID 59, Seq. ID 60, Seq. ID 61, Seq. ID 62, Seq. ID 63, and Seq. ID 64 respectively.

In one of the embodiments, the present invention provides an antibody or antigen-binding fragment which specifically binds to human PD-L2 comprising a light chain of Seq. ID 49 consisting of LCDR1, LCDR2, and LCDR3, respectively, set forth in Seq. ID Nos. 1, 2, and 3; and a heavy chain of Seq. ID 57 consisting of HCDR1, HCDR2, and HCDR3, respectively, set forth in Seq. ID Nos. 4, 5, and 6. Said antibody specifically binds to human PD-L2 with an EC50 of about 28.38 nM.

In one of the embodiments, the present invention provides an antibody or antigen-binding fragment which specifically binds to human PD-L2 comprising a light chain of Seq. ID 50 consisting of LCDR1, LCDR2, and LCDR3, respectively, set forth in Seq. ID Nos. 7, 8, and 9; and a heavy chain of Seq. ID 58 consisting of HCDR1, HCDR2, and HCDR3, respectively, set forth in Seq. ID Nos. 10, 11 and 12. Said antibody specifically binds to human PD-L2 with an EC50 of about 8.159 nM.

In one of the embodiments, the present invention provides an antibody or antigen-binding fragment which specifically binds to human PD-L2 comprising a light chain of Seq. ID 51 consisting of LCDR1, LCDR2, and LCDR3, respectively, set forth in Seq. ID Nos. 13, 14, and 15; and a heavy chain of Seq. ID 59 consisting of HCDR1, HCDR2, and HCDR3, respectively, set forth in Seq. ID Nos. 16, 17, and 18. Said antibody specifically binds to human PD-L2 with an EC50 of about 11.64 nM.

In one of the embodiments, the present invention provides an antibody or antigen-binding fragment which specifically binds to human PD-L2 comprising a light chain of Seq. ID 52 consisting of LCDR1, LCDR2, and LCDR3, respectively, set forth in Seq. ID Nos. 19, 20, and 21; and a heavy chain of Seq. ID 60 consisting of HCDR1, HCDR2, and HCDR3, respectively, set forth in Seq. ID Nos. 22, 23, and 24. Said antibody specifically binds to human PD-L2 with an EC50 of about 37.66 nM.

In one of the embodiments, the present invention provides an antibody or antigen-binding fragment which specifically binds to human PD-L2 comprising a light chain of Seq. ID 53 consisting of LCDR1, LCDR2, and LCDR3, respectively, set forth in Seq. ID Nos. 25, 26, and 27; and a heavy chain of Seq. ID 61 consisting of HCDR1, HCDR2, and HCDR3, respectively, set forth in Seq. ID Nos. 28, 29, and 30. Said antibody specifically binds to human PD-L2 with an EC50 of about 11.18 nM.

In one of the embodiments, the present invention provides an antibody or antigen-binding fragment which specifically binds to human PD-L2 comprising a light chain of Seq. ID 54 consisting of LCDR1, LCDR2, and LCDR3, respectively, set forth in Seq. ID Nos. 31, 32, and 33; and a heavy chain of Seq. ID 62 consisting of HCDR1, HCDR2, and HCDR3, respectively, set forth in Seq. ID Nos. 34, 35, and 36. Said antibody specifically binds to human PD-L2 with an EC50 of about 15.83 nM.

In one of the embodiments, the present invention provides an antibody or antigen-binding fragment which specifically binds to human PD-L2 comprising a light chain of Seq. ID 55 consisting of LCDR1, LCDR2, and LCDR3, respectively, set forth in Seq. ID Nos. 37, 38, and 39; and a heavy chain of Seq. ID 63 consisting of HCDR1, HCDR2, and HCDR3, respectively, set forth in Seq. ID Nos. 40, 41 and 42. Said antibody specifically binds to human PD-L2 with an EC50 of about 28.32 nM.

In one of the embodiments, the present invention provides a human antibody or antigen-binding fragment which specifically binds to human PD-L2 comprising a light chain of Seq. ID 56 consisting of LCDR1, LCDR2, and LCDR3, respectively, set forth in Seq. ID Nos. 43, 44, and 45; and a heavy chain of Seq. ID 64 consisting of HCDR1, HCDR2, and HCDR3, respectively, set forth in Seq. ID Nos. 46, 47, and 48. Said antibody specifically binds to human PD-L2 with an EC50 of about 32.41 nM.

### BRIEF DESCRIPTION OF THE DRAWINGS

FIG. 1A-D depicts graphical representation of FACS blocking results of 36 candidate antibodies on huPD-L2-HEK293 cells compared to negative control antibody R15-F7 (lgG1), and reference antibody or positive control 24F.10C12.

FIG. 2 depicts a graphical representation of luciferace assay results for 7 candidate molecules, A57, A96, A115, A117, A125, A131, A133, and A154 on the PD-1-NF-AT luciferase-Jurkat reporter system compared to negative control antibody R15-F7 (lgG1), and reference antibody or positive control 24F.10C12.

FIG. 3 depicts a graphical representation of tumour growth/volume in mice upto 30 days post tumour cells inoculation and administration of 3 candidate antibody (A96, A125, and A131) at 2 doses 2 mpk or 5 mpk as listed in Table 6, compared to negative control antibody R15-F7 (lgG1), and reference antibody or positive control 24F.10C12.

FIG. 4 depicts a graphical representation of mice weight over 25-30 days after tumour cell inoculation and administration of 3 candidate antibody (A96, A125, and A131) at 5 mpk dose as listed in Table 6, compared to negative control antibody R15-F7 (lgG1), and reference antibody or positive control 24F.10C12.

FIG. 5 depicts a graphical representation of tumour weight at Day 30 in mice after tumour cell inoculation and administration of A96, A125, A131, 24F.10C12, and R15-F7 (lgG1) using 5 mpk dosage.

### DETAILED DESCRIPTION OF THE INVENTION

### Definitions

The term “antibody” as used herein includes any immunoglobulin, monoclonal antibody, polyclonal antibody that binds to a specific antigen. A native intact antibody comprises two heavy chains (H) and two light chains (L). Each heavy chain consists of a variable region (V_{H}) and a first, second, and third constant region (C_{H}), while each light chain consists of a variable region (V_{L}) and a constant region (C_{L}). The antibody has a “Y” shape, with the stem of the Y consisting of the second and third constant regions of two heavy chains bound together via disulfide bonding. Each arm of the Y includes the variable region and first constant region of a single heavy chain bound to the variable and constant regions of a single light chain. The variable regions of the light and heavy chains are responsible for antigen binding. The constant regions of the heavy and light chains are not involved in antigen binding, but exhibit various effector functions.

The term “CDR or complementarity determining regions” as used herein includes parts of variable region in both chains of antibody. These generally are three highly variable loops in each of the light and heavy chains. Light (L) chain CDRs include LCDR1, LCDR2, and LCDR3, and heavy (H) chain CDRs include HCDR1, HCDR2, HCDR3).

The term “antigen-binding fragment” as used herein refers to an antibody fragment formed from a portion of an antibody comprising one or more CDRs, or any other antibody fragment that binds to an antigen but does not comprise an intact native antibody structure. An antigen-binding fragment is capable of binding to the same antigen to which the parent antibody binds.

The term “fully human antibody” or “human antibody” as used herein, with reference to antibody or antigen-binding fragment, means that the antibody or the antigen-binding fragment has or consists of amino acid sequence(s) corresponding to that of an antibody produced by a human or a human immune cell, or derived from a non-human source such as a transgenic non-human animal that utilizes human antibody repertoires or other human antibody-encoding sequences. In certain embodiments, a fully human antibody does not comprise amino acid residues (in particular antigen-binding residues) derived from a non-human antibody.

The term “humanized antibody” as used herein, with reference to antibody or antigen-binding fragment, means that the antibody is derived from non-human species whose amino acid sequences have been modified to increase their similarity to antibody variants produced naturally in humans.

The term “monoclonal antibody” as used herein refers to an antibody obtained from a population of substantially homogeneous antibodies, i.e., the individual antibodies comprising the population are identical except for possible naturally occurring mutations and/or post-translation modifications (e.g., isomerizations, amidations) that may be present in minor amounts. Monoclonal antibodies are highly specific, being directed against a single antigenic site. In contrast to polyclonal antibody preparations which typically include different antibodies directed against different determinants (epitopes), each monoclonal antibody is directed against a single determinant on the antigen.

The term “PD-L2” as used herein refers to programmed cell death ligand 2 also as CD273, B7-DC, PDCD1L2, PDCD1LG2, PDL2. PD-L2 is a known transmembrane protein expressed on various tumour cells, stromal cells or both, and binds to PD-1.

The term “PD-1”, as used herein refers to programmed cell death-1 also called as CD279, is known as a key immune-checkpoint receptor expressed by activated T cells, which mediates immunosuppression. Inhibition of the interaction between PD-1 and PD-L1 can enhance T-cell responses and thus mediates anti-cancer activity.

The term “Anti-PD-L2 antibody” as used herein refers to an antibody that is capable of specific binding to PD-L2 (e.g. human PD-L2) with an affinity which is sufficient to provide for diagnostic and/or therapeutic use.

The ability to “block binding” as used herein refers to the ability of an antibody or antigen-binding fragment to inhibit the binding interaction between two molecules (e.g. human PD-L2 and an anti-PD-L2 antibody) to any detectable degree.

The term “EC50” as used herein refers to the concentration of antibody required to a block binding of PD-1/PD-L2 by 50% of the maximum binding.

The term “vector” as used herein refers to a vehicle into which a polynucleotide encoding a protein may be operably inserted so as to bring about the expression of that protein. A vector may be used to transform, transduce, or transfect a host cell so as to bring about expression of the genetic element it carries within the host cell. Examples of vectors include plasmids, phagemids, cosmids, artificial chromosomes such as yeast artificial chromosome (YAC), bacterial artificial chromosome (BAC), or P1-derived artificial chromosome (PAC), bacteriophages such as lambda phage or M13 phage, and animal viruses. A vector may contain a variety of elements for controlling expression, including promoter sequences, transcription initiation sequences, enhancer sequences, selectable elements, and reporter genes. In addition, the vector may contain an origin of replication. A vector may also include materials to aid in its entry into the cell, including but not limited to a viral particle, a liposome, or a protein coating.

The present invention now will be described hereinafter with reference to the detailed description, in which some, but not all embodiments of the invention are indicated. Indeed, the invention may be embodied in many different forms and should not be construed as limited to the embodiments set forth herein; rather, these embodiments are provided so that this disclosure will satisfy applicable legal requirements. The present invention is described fully herein with non-limiting embodiments and exemplary experimentation.

In the main embodiment of the present invention, the invention provides human antibodies or antigen-binding fragments which specifically binds to human PD-L2. Further, the present invention provides CDRs of the light chain and heavy chain of each human antibody or antigen-binding fragment which specifically bind to human PD-L2. The amino acid sequences of exemplary antibodies and the CDRs are set forth in the Table 1.

TABLE 1
List of polypeptide sequences of anti-PD-L2 antibody light chains, heavy
chains, and the CDRs of light and heavy chains

| | | |
|---|---|---|
| Name | Seq. ID | Amino acid sequence |

LCDR1

| | | |
|---|---|---|
| A57-LCDR1 | 1 | SSNVGTTYVSW |
| A96-LCDR1 | 7 | QSVSSNLAW |
| A115- | 13 | QSVSSNLAW |
| LCDR1 | | 100% identity with Seq. ID 7 |
| A117- | 19 | SSNIGNNYVSW |
| LCDR1 | | |
| A125- | 25 | QGISSWLAW |
| LCDR1 | | |
| A131- | 31 | QGVDSSLNW |
| LCDR1 | | |
| A133- | 37 | QGIETDLAW |
| LCDR1 | | |
| A154- | 43 | QSVSSYLAW |
| LCDR1 | | |

LCDR2

| | | |
|---|---|---|
| A57-LCDR2 | 2 | PKLLIYSNNQRPS |
| A96-LCDR2 | 8 | PRLLIYGASTRAT |
| A115- | 14 | PRLLIYDASNRAT |
| LCDR2 | | |
| A117- | 20 | PKLLIYDNNKRPS |
| LCDR2 | | |
| A125- | 26 | PKLLIYAASSLQS |
| LCDR2 | | |
| A131- | 32 | PRLLIYDASNRAT |
| LCDR2 | | |
| A133- | 38 | PKRLIYGASTSQN |
| LCDR2 | | |
| A154- | 44 | PRLLIYDASNRAT |
| LCDR2 | | 100% identity with Seq. ID 14 |

LCDR3

| | | |
|---|---|---|
| A57-LCDR3 | 3 | AAWDDSLNGRVFGGGT |
| A96-LCDR3 | 9 | QHQGTFGQGT |
| A115- | 15 | QQRNNWPITFGQGT |
| LCDR3 | | |
| A117- | 21 | GTWDSSLSAWVFGGGT |
| LCDR3 | | |
| A125- | 27 | QQANSFPLTFGGGT |
| LCDR3 | | |
| A131- | 33 | QQRNNWPLTFGGGT |
| LCDR3 | | |
| A133- | 39 | LQHNNFPYTFGQGT |
| LCDR3 | | |
| A154- | 45 | QQRSNWPLTFGGGT |
| LCDR3 | | |

HCDR1

| | | |
|---|---|---|
| A57-HCDR1 | 4 | SGGTFSSYAI |
| | | 100% identity with Seq. ID 10 |
| A96-HCDR1 | 10 | SGGTFSSYAI |
| A115- | 16 | SGGTFSSYAI |
| HCDR1 | | 100% identity with Seq. ID 10 |
| A117- | 22 | SGGTFSSYAI |
| HCDR1 | | 100% identity with Seq. ID 10 |
| A125- | 28 | SGGTFSSYAI |
| HCDR1 | | 100% identity with Seq. ID 10 |
| A131- | 34 | SGGTFSSYAI |
| HCDR1 | | 100% identity with Seq. ID 10 |
| A133- | 40 | SGGTFSSYAI |
| HCDR1 | | 100% identity with Seq. ID 10 |
| A154- | 46 | SGGTFSSYAI |
| HCDR1 | | 100% identity with Seq. ID 10 |

HCDR2

| | | |
|---|---|---|
| A57-HCDR2 | 5 | LEWMGRIIPILGIANY |
| | | 100% identity with Seq. ID 17 |
| A96-HCDR2 | 11 | LEWMGGIIPIFGTANY |
| A115- | 17 | LEWMGRIIPILGIANY |
| HCDR2 | | |
| A117- | 23 | LEWMGRIIPILGIANY |
| HCDR2 | | 100% identity with Seq. ID 17 |
| A125- | 29 | LEWMGRIIPILGIANY |
| HCDR2 | | 100% identity with Seq. ID 17 |
| A131- | 35 | LEWMGRIIPILGIANY |
| HCDR2 | | |
| A133- | 41 | LEWMGRIIPILGIANY |
| HCDR2 | | 100% identity with Seq. ID 17 |
| A154- | 47 | LEWMGRIIPILGIANY |
| HCDR2 | | 100% identity with Seq. ID 17 |

HCDR3

| | | |
|---|---|---|
| A57-HCDR3 | 6 | VVPAYYYGMDVW |
| A96-HCDR3 | 12 | VRRGYYAFDIW |
| A115- | 18 | EPSSSGYPSGDYW |
| HCDR3 | | |
| A117- | 24 | MRGDTANFDYW |
| HCDR3 | | |
| A125- | 30 | DFGGYYSGAFDIW |
| HCDR3 | | |
| A131- | 36 | EPSSSGYPSGDYW |
| HCDR3 | | |
| A133- | 42 | DQRRGGKVGMDVW |
| HCDR3 | | |
| A154- | 48 | EPPYSGSYSYGMDVW |
| HCDR3 | | |

Light Chains

| | | |
|---|---|---|
| A57-LC | 49 | QSVLTQPPSVSAAPGQTVTISCSGSSSNVGTTYVSWYQQLP |
| | | GTAPKLLIYSNNQRPSGVPDRFSGSKSGTSASLAISGLQSED |
| | | EADYYCAAWDDSLNGRVFGGGTKLTVLGQPKAAPSVTLF |
| | | PPSSEELQANKATLVCLISDFYPGAVTVAWKADSSPVKAG |
| | | VETTTPSKQSNNKYAASSYLSLTPEQWKSHRSYSCQVTHE |
| | | GSTVEKTVAPTECS |
| A96-LC | 50 | EIVMTQSPATLSVSPGERATLSCRASQSVSSNLAWYQQKPG |
| | | QAPRLLIYGASTRATGIPARFSGSGSGTEFTLTISSLQSEDFA |
| | | VYYCQHQGTFGQGTKVEIKRTVAAPSVFIFPPSDEQLKSGT |
| | | ASVVCLLNNFYPREAKVQWKVDNALQSGNSQESVTEQDS |
| | | KDSTYSLSSTLTLSKADYEKHKVYACEVTHQGLSSPVTKSF |
| | | NRGEC |
| A115-LC | 51 | EIVLTQSPATLSLSPGERATLSCRASQSVSSNLAWYQQKPG |
| | | QAPRLLIYDASNRATGIPARFSGSGSATDFTLTISSLEPEDFA |
| | | VYYCQQRNNWPITFGQGTRLEIKRTVAAPSVFIFPPSDEQL |
| | | KSGTASVVCLLNNFYPREAKVQWKVDNALQSGNSQESVT |
| | | EQDSKDSTYSLSSTLTLSKADYEKHKVYACEVTHQGLSSP |
| | | VTKSFNRGEC |
| A117-LC | 52 | QSVLTQPPSVSAAPGQKVTISCSGSSSNIGNNYVSWYQQLP |
| | | GTAPKLLIYDNNKRPSGIPDRESGSKSGTSATLGITGLQTGD |
| | | EADYYCGTWDSSLSAWVFGGGTKLTVLGQPKAAPSVTLF |
| | | PPSSEELQANKATLVCLISDFYPGAVTVAWKADSSPVKAG |
| | | VETTTPSKQSNNKYAASSYLSLTPEQWKSHRSYSCQVTHE |
| | | GSTVEKTVAPTECS |
| A125-LC | 53 | DIQMIQSPSSVSASVGDRVTITCRASQGISSWLAWYQQKPG |
| | | KAPKLLIYAASSLQSGVPSRFSGSGSGTDFTLTISSLQPEDFA |
| | | TYYCQQANSFPLTFGGGTKVEIKRTVAAPSVFIFPPSDEQLK |
| | | SGTASVVCLLNNFYPREAKVQWKVDNALQSGNSQESVTE |
| | | QDSKDSTYSLSSTLTLSKADYEKHKVYACEVTHQGLSSPV |
| | | TKSFNRGEC |
| A131-LC | 54 | EIVLTQSPAALSLSPGERATLSCRASQGVDSSLNWYQQKPG |
| | | QAPRLLIYDASNRATGIPARFSGSGSGTDFTLTISSLEPEDFA |
| | | VYYCQQRNNWPLTFGGGTKVEIKRTVAAPSVFIFPPSDEQL |
| | | KSGTASVVCLLNNFYPREAKVQWKVDNALQSGNSQESVT |
| | | EQDSKDSTYSLSSTLTLSKADYEKHKVYACEVTHQGLSSP |
| | | VTKSFNRGEC |
| A133-LC | 55 | NIQLTQSPSSLSASVGDRVTITCRASQGIETDLAWYQQKPG |
| | | KAPKRLIYGASTSQNGVPSRFSGGGYGTHFTLTISGLQPEDF |
| | | ATYYCLQHNNFPYTFGQGTRLEIKRTVAAPSVFIFPPSDEQL |
| | | KSGTASVVCLLNNFYPREAKVQWKVDNALQSGNSQESVT |
| | | EQDSKDSTYSLSSTLTLSKADYEKHKVYACEVTHQGLSSP |
| | | VTKSFNRGEC |
| A154-LC | 56 | EIVMTQSPATLSLSPGERATLSCRASQSVSSYLAWYQQKPG |
| | | QAPRLLIYDASNRATGIPARFSGSGSGTDFTLTISSLEPEDFA |
| | | VYYCQQRSNWPLTFGGGTKVEIKRTVAAPSVFIFPPSDEQL |
| | | KSGTASVVCLLNNFYPREAKVQWKVDNALQSGNSQESVT |
| | | EQDSKDSTYSLSSTLTLSKADYEKHKVYACEVTHQGLSSP |
| | | VTKSFNRGEC |

Heavy Chains

| | | |
|---|---|---|
| A57-HC | 57 | QVQLVQSGAEVKKPGSSVKVSCKASGGTFSSYAISWVRQA |
| | | PGQGLEWMGRIIPILGIANYAQKFQGRVTITADKSTSTAYM |
| | | ELSSLRSEDTAVYYCARVVPAYYYGMDVWGQGTTVTVSS |
| | | ASTKGPSVFPLAPSSKSTSGGTAALGCLVKDYFPEPVTVSW |
| | | NSGALTSGVHTFPAVLQSSGLYSLSSVVTVPSSSLGTQTYIC |
| | | NVNHKPSNTKVDKKVEPKSCDKTHTCPPCPAPELLGGPSV |
| | | FLFPPKPKDTLMISRTPEVTCVVVDVSHEDPEVKFNWYVD |
| | | GVEVHNAKTKPREEQYNSTYRVVSVLTVLHQDWLNGKEY |
| | | KCKVSNKALPAPIEKTISKAKGQPREPQVYTLPPSRDELTK |
| | | NQVSLTCLVKGFYPSDIAVEWESNGQPENNYKTTPPVLDS |
| | | DGSFFLYSKLTVDKSRWQQGNVFSCSVMHEALHNHYTQK |
| | | SLSLSPGK |
| A96-HC | 58 | QVQLVQSGAEVKKPGSSVKVSCKASGGTFSSYAISWVRQA |
| | | PGQGLEWMGGIIPIFGTANYAQKFQGRVTITADESTSTAYM |
| | | ELSSLRSEDTAVYYCARVRRGYYAFDIWGQGTMVTVSSAS |
| | | TKGPSVFPLAPSSKSTSGGTAALGCLVKDYFPEPVTVSWNS |
| | | GALTSGVHTFPAVLQSSGLYSLSSVVTVPSSSLGTQTYICN |
| | | VNHKPSNTKVDKKVEPKSCDKTHTCPPCPAPELLGGPSVFL |
| | | FPPKPKDTLMISRTPEVTCVVVDVSHEDPEVKFNWYVDGV |
| | | EVHNAKTKPREEQYNSTYRVVSVLTVLHQDWLNGKEYKC |
| | | KVSNKALPAPIEKTISKAKGQPREPQVYTLPPSRDELTKNQ |
| | | VSLTCLVKGFYPSDIAVEWESNGQPENNYKTTPPVLDSDGS |
| | | FFLYSKLTVDKSRWQQGNVFSCSVMHEALHNHYTQKSLS |
| | | LSPGK |
| A115-HC | 59 | QVQLQQSGAEVKKPGSSVKVSCKASGGTFSSYAISWVRQA |
| | | PGQGLEWMGRIIPILGIANYAQKFQGRVTITADKSTSTAYM |
| | | ELSSLRSEDTAVYYCAREPSSSGYPSGDYWGQGTLVTVSS |
| | | ASTKGPSVFPLAPSSKSTSGGTAALGCLVKDYFPEPVTVSW |
| | | NSGALTSGVHTFPAVLQSSGLYSLSSVVTVPSSSLGTQTYIC |
| | | NVNHKPSNTKVDKKVEPKSCDKTHTCPPCPAPELLGGPSV |
| | | FLFPPKPKDTLMISRTPEVTCVVVDVSHEDPEVKFNWYVD |
| | | GVEVHNAKTKPREEQYNSTYRVVSVLTVLHQDWLNGKEY |
| | | KCKVSNKALPAPIEKTISKAKGQPREPQVYTLPPSRDELTK |
| | | NQVSLTCLVKGFYPSDIAVEWESNGQPENNYKTTPPVLDS |
| | | DGSFFLYSKLTVDKSRWQQGNVFSCSVMHEALHNHYTQK |
| | | SLSLSPGK |
| A117-HC | 60 | QVQLVQSGAEVKKPGSSVKVSCKASGGTFSSYAISWVRQA |
| | | PGQGLEWMGRIIPILGIANYAQKFQGRVTITADKSTSTAYM |
| | | ELSSLRSEDTAVYYCARMRGDTANFDYWGQGTLVTVSSA |
| | | STKGPSVFPLAPSSKSTSGGTAALGCLVKDYFPEPVTVSWN |
| | | SGALTSGVHTFPAVLQSSGLYSLSSVVTVPSSSLGTQTYICN |
| | | VNHKPSNTKVDKKVEPKSCDKTHTCPPCPAPELLGGPSVFL |
| | | FPPKPKDTLMISRTPEVTCVVVDVSHEDPEVKFNWYVDGV |
| | | EVHNAKTKPREEQYNSTYRVVSVLTVLHQDWLNGKEYKC |
| | | KVSNKALPAPIEKTISKAKGQPREPQVYTLPPSRDELTKNQ |
| | | VSLTCLVKGFYPSDIAVEWESNGQPENNYKTTPPVLDSDGS |
| | | FFLYSKLTVDKSRWQQGNVFSCSVMHEALHNHYTQKSLS |
| | | LSPGK |
| A125-HC | 61 | QVQLVQSGAEVKKPGSSVKVSCKASGGTFSSYAISWVRQA |
| | | PGQGLEWMGRIIPILGIANYAQKFQGRVTITADKSTSTAYM |
| | | ELSSLRSEDTAVYYCARDFGGYYSGAFDIWGQGTMVTVSS |
| | | ASTKGPSVFPLAPSSKSTSGGTAALGCLVKDYFPEPVTVSW |
| | | NSGALTSGVHTFPAVLQSSGLYSLSSVVTVPSSSLGTQTYIC |
| | | NVNHKPSNTKVDKKVEPKSCDKTHTCPPCPAPELLGGPSV |
| | | FLFPPKPKDTLMISRTPEVTCVVVDVSHEDPEVKFNWYVD |
| | | GVEVHNAKTKPREEQYNSTYRVVSVLTVLHQDWLNGKEY |
| | | KCKVSNKALPAPIEKTISKAKGQPREPQVYTLPPSRDELTK |
| | | NQVSLTCLVKGFYPSDIAVEWESNGQPENNYKTTPPVLDS |
| | | DGSFFLYSKLTVDKSRWQQGNVFSCSVMHEALHNHYTQK |
| | | SLSLSPGK |
| A131-HC | 62 | QVQLQQSGAEVKKPGSSVKVSCKASGGTFSSYAISWVRQA |
| | | PGQGLEWMGRIIPILGIANYAQKFQGRVTITADKSTSTAYM |
| | | ELSSLRSEDTAVYYCAREPSSSGYPSGDYWGQGTLVTVSS |
| | | ASTKGPSVFPLAPSSKSTSGGTAALGCLVKDYFPEPVTVSW |
| | | NSGALTSGVHTFPAVLQSSGLYSLSSVVTVPSSSLGTQTYIC |
| | | NVNHKPSNTKVDKKVEPKSCDKTHTCPPCPAPELLGGPSV |
| | | FLFPPKPKDTLMISRTPEVTCVVVDVSHEDPEVKFNWYVD |
| | | GVEVHNAKTKPREEQYNSTYRVVSVLTVLHQDWLNGKEY |
| | | KCKVSNKALPAPIEKTISKAKGQPREPQVYTLPPSRDELTK |
| | | NQVSLTCLVKGFYPSDIAVEWESNGQPENNYKTTPPVLDS |
| | | DGSFFLYSKLTVDKSRWQQGNVFSCSVMHEALHNHYTQK |
| | | SLSLSPGK |
| A133-HC | 63 | EVQLVQSGAEVKKPGSSVKVSCKASGGTFSSYAISWVRQA |
| | | PGQGLEWMGRIIPILGIANYAQKFQGRVTITADKSTSTAYM |
| | | ELSSLRSEDTAVYYCARDQRRGGKVGMDVWGQGTLVTVS |
| | | SASTKGPSVFPLAPSSKSTSGGTAALGCLVKDYFPEPVTVS |
| | | WNSGALTSGVHTFPAVLQSSGLYSLSSVVTVPSSSLGTQTY |
| | | ICNVNHKPSNTKVDKKVEPKSCDKTHTCPPCPAPELLGGPS |
| | | VFLFPPKPKDTLMISRTPEVTCVVVDVSHEDPEVKFNWYV |
| | | DGVEVHNAKTKPREEQYNSTYRVVSVLTVLHQDWLNGKE |
| | | YKCKVSNKALPAPIEKTISKAKGQPREPQVYTLPPSRDELT |
| | | KNQVSLTCLVKGFYPSDIAVEWESNGQPENNYKTTPPVLD |
| | | SDGSFFLYSKLTVDKSRWQQGNVFSCSVMHEALHNHYTQ |
| | | KSLSLSPGK |
| A154-HC | 64 | QVQLVQSGAEVKKPGSSVKVSCKASGGTFSSYAISWVRQA |
| | | PGQGLEWMGRIIPILGIANYAQKFQGRVTITADKSTSTAYM |
| | | ELSSLRSEDTAVYYCAREPPYSGSYSYGMDVWGQGTMVT |
| | | VSSASTKGPSVFPLAPSSKSTSGGTAALGCLVKDYFPEPVT |
| | | VSWNSGALTSGVHTFPAVLQSSGLYSLSSVVTVPSSSLGTQ |
| | | TYICNVNHKPSNTKVDKKVEPKSCDKTHTCPPCPAPELLG |
| | | GPSVFLFPPKPKDTLMISRTPEVTCVVVDVSHEDPEVKFNW |
| | | YVDGVEVHNAKTKPREEQYNSTYRVVSVLTVLHQDWLN |
| | | GKEYKCKVSNKALPAPIEKTISKAKGQPREPQVYTLPPSRD |
| | | ELTKNQVSLTCLVKGFYPSDIAVEWESNGQPENNYKTTPP |
| | | VLDSDGSFFLYSKLTVDKSRWQQGNVFSCSVMHEALHNH |
| | | YTQKSLSLSPGK |

In another embodiment of the present invention, the invention provides nucleic acid sequences encoding human antibodies or antigen-binding fragments which specifically bind to human PD-L2. Further, the present invention provides nucleic acid sequences encoding the light chain and heavy chain of each human antibody or antigen-binding fragment which specifically bind to human PD-L2. The nucleic acid sequences encoding the light chains and heavy chains of exemplary antibodies are set forth in the Table 2.

TABLE 2
List of nucleic acid sequences encoding light chains and heavy chains of
anti-PD-L2 antibody

| | | |
|---|---|---|
| | Seq. | |
| Name | ID | Nucleic Acid sequences |

Light Chains

| | | |
|---|---|---|
| A57- | 65 | CAGTCTGTCTTGACGCAGCCGCCCTCAGTGTCTGCGGCCCCAGGA |
| LC | | CAGACGGTCACCATCTCCTGCTCTGGAAGCAGCTCCAACGTTGGG |
| | | ACTACTTATGTATCCTGGTACCAGCAGCTCCCAGGAACGGCCCCC |
| | | AAACTCCTCATCTATAGTAATAATCAGCGGCCCTCAGGGGTCCCT |
| | | GACCGATTCTCTGGCTCCAAGTCTGGCACCTCAGCCTCCCTGGCC |
| | | ATCAGTGGGCTCCAGTCTGAGGATGAGGCTGATTATTACTGTGCA |
| | | GCATGGGATGACAGCCTGAATGGTCGAGTGTTCGGCGGAGGGAC |
| | | CAAGCTGACCGTCCTAGGACAGCCTAAGGCTGCCCCTTCCGTGAC |
| | | ACTGTTCCCTCCATCCTCTGAGGAACTGCAGGCCAACAAGGCTAC |
| | | CCTCGTGTGCCTGATCTCCGACTTTTACCCTGGCGCTGTGACCGTG |
| | | GCCTGGAAGGCTGATAGTTCTCCTGTGAAGGCCGGCGTGGAAAC |
| | | CACCACACCTTCCAAGCAGTCCAACAACAAATACGCCGCCTCCTC |
| | | CTACCTGTCTCTGACCCCTGAACAGTGGAAGTCCCACCGGTCCTA |
| | | CAGCTGCCAAGTGACCCATGAGGGCTCCACCGTGGAAAAGACCG |
| | | TGGCTCCTACCGAGTGCTCT |
| A96- | 66 | GAAATTGTGATGACGCAGTCTCCAGCCACCCTGTCTGTGTCTCCA |
| LC | | GGGGAAAGAGCCACCCTCTCCTGCAGGGCCAGTCAGAGTGTTAG |
| | | CAGCAACTTAGCCTGGTACCAGCAGAAACCTGGCCAGGCTCCCA |
| | | GGCTCCTCATCTATGGTGCATCCACCAGGGCCACTGGTATCCCAG |
| | | CCAGGTTCAGTGGCAGTGGGTCTGGGACAGAGTTCACTCTCACCA |
| | | TCAGCAGCCTGCAGTCTGAAGATTTTGCAGTTTATTACTGTCAGC |
| | | ACCAGGGGACGTTCGGCCAAGGGACCAAGGTGGAGATCAAAAGG |
| | | ACCGTGGCTGCCCCCAGCGTGTTCATCTTCCCTCCTAGCGACGAG |
| | | CAGCTGAAGAGCGGCACCGCTAGCGTGGTGTGTCTGCTGAATAA |
| | | CTTCTATCCCAGGGAGGCCAAGGTGCAGTGGAAGGTGGATAACG |
| | | CCCTGCAGAGCGGCAACTCCCAGGAGTCCGTGACCGAGCAGGAC |
| | | TCCAAGGACAGCACCTACTCCCTGAGCTCCACCCTGACCCTGTCC |
| | | AAGGCTGATTATGAGAAGCACAAGGTGTATGCTTGCGAGGTGAC |
| | | ACACCAGGGCCTGTCCAGCCCTGTGACCAAGAGCTTCAACCGGG |
| | | GCGAGTGC |
| A115- | 67 | GAAATTGTGTTGACACAGTCTCCAGCCACCCTGTCTTTGTCTCCA |
| LC | | GGGGAAAGAGCCACCCTCTCCTGTAGGGCCAGTCAGAGTGTTAG |
| | | CAGCAACTTAGCCTGGTACCAACAGAAACCTGGCCAGGCTCCCA |
| | | GACTCCTCATCTATGATGCATCCAACAGGGCCACTGGCATCCCAG |
| | | CCAGGTTCAGTGGCAGTGGGTCTGCGACAGACTTCACTCTCACCA |
| | | TCAGCAGCCTAGAACCTGAAGATTTTGCAGTTTATTACTGTCAGC |
| | | AGCGTAACAACTGGCCGATCACCTTCGGCCAAGGGACACGACTG |
| | | GAGATTAAAAGGACCGTGGCTGCCCCCAGCGTGTTCATCTTCCCT |
| | | CCTAGCGACGAGCAGCTGAAGAGCGGCACCGCTAGCGTGGTGTG |
| | | TCTGCTGAATAACTTCTATCCCAGGGAGGCCAAGGTGCAGTGGAA |
| | | GGTGGATAACGCCCTGCAGAGCGGCAACTCCCAGGAGTCCGTGA |
| | | CCGAGCAGGACTCCAAGGACAGCACCTACTCCCTGAGCTCCACCC |
| | | TGACCCTGTCCAAGGCTGATTATGAGAAGCACAAGGTGTATGCTT |
| | | GCGAGGTGACACACCAGGGCCTGTCCAGCCCTGTGACCAAGAGC |
| | | TTCAACCGGGGCGAGTGC |
| A117- | 68 | CAGTCTGTGCTGACGCAGCCGCCCTCAGTGTCTGCGGCCCCAGGA |
| LC | | CAGAAGGTCACCATCTCCTGCTCTGGAAGCAGCTCCAACATTGGG |
| | | AATAATTATGTATCCTGGTACCAGCAGCTCCCAGGAACAGCCCCC |
| | | AAACTCCTCATTTATGACAATAATAAGCGACCCTCAGGGATTCCT |
| | | GACCGATTCTCTGGCTCCAAGTCTGGCACGTCAGCCACCCTGGGC |
| | | ATCACCGGACTCCAGACTGGGGACGAGGCCGATTATTACTGCGG |
| | | AACATGGGATAGCAGCCTGAGTGCTTGGGTGTTCGGCGGAGGGA |
| | | CCAAGCTGACCGTCCTAGGACAGCCTAAGGCTGCCCCTTCCGTGA |
| | | CACTGTTCCCTCCATCCTCTGAGGAACTGCAGGCCAACAAGGCTA |
| | | CCCTCGTGTGCCTGATCTCCGACTTTTACCCTGGCGCTGTGACCGT |
| | | GGCCTGGAAGGCTGATAGTTCTCCTGTGAAGGCCGGCGTGGAAA |
| | | CCACCACACCTTCCAAGCAGTCCAACAACAAATACGCCGCCTCCT |
| | | CCTACCTGTCTCTGACCCCTGAACAGTGGAAGTCCCACCGGTCCT |
| | | ACAGCTGCCAAGTGACCCATGAGGGCTCCACCGTGGAAAAGACC |
| | | GTGGCTCCTACCGAGTGCTCT |
| A125- | 69 | GACATCCAGATGATCCAGTCTCCATCTTCCGTGTCTGCATCTGTA |
| LC | | GGAGACAGAGTCACCATCACTTGTCGGGCGAGTCAGGGTATTAG |
| | | CAGCTGGTTAGCCTGGTATCAGCAGAAACCAGGGAAAGCCCCTA |
| | | AGCTCCTGATCTATGCTGCATCCAGTTTGCAAAGTGGGGTCCCAT |
| | | CAAGGTTCAGCGGCAGTGGATCTGGGACAGATTTCACTCTCACCA |
| | | TCAGCAGCCTGCAGCCTGAAGATTTTGCAACTTACTATTGTCAAC |
| | | AGGCTAACAGTTTCCCTCTCACTTTCGGCGGAGGGACCAAGGTGG |
| | | AGATCAAAAGGACCGTGGCTGCCCCCAGCGTGTTCATCTTCCCTC |
| | | CTAGCGACGAGCAGCTGAAGAGCGGCACCGCTAGCGTGGTGTGT |
| | | CTGCTGAATAACTTCTATCCCAGGGAGGCCAAGGTGCAGTGGAA |
| | | GGTGGATAACGCCCTGCAGAGCGGCAACTCCCAGGAGTCCGTGA |
| | | CCGAGCAGGACTCCAAGGACAGCACCTACTCCCTGAGCTCCACCC |
| | | TGACCCTGTCCAAGGCTGATTATGAGAAGCACAAGGTGTATGCTT |
| | | GCGAGGTGACACACCAGGGCCTGTCCAGCCCTGTGACCAAGAGC |
| | | TTCAACCGGGGCGAGTGC |
| A131- | 70 | GAAATTGTGTTGACACAGTCTCCAGCCGCCCTGTCTTTGTCTCCA |
| LC | | GGGGAAAGAGCCACCCTCTCCTGCAGGGCCAGTCAGGGTGTTGA |
| | | CAGCTCCTTAAACTGGTACCAACAGAAACCTGGCCAGGCTCCCAG |
| | | GCTCCTCATCTATGATGCATCCAACAGGGCCACTGGCATCCCAGC |
| | | CAGGTTCAGTGGCAGTGGGTCTGGGACAGACTTCACTCTCACCAT |
| | | CAGCAGCCTAGAGCCTGAAGATTTTGCAGTTTATTACTGTCAGCA |
| | | GCGTAACAACTGGCCTCTCACTTTCGGCGGAGGGACCAAGGTGG |
| | | AGATCAAAAGGACCGTGGCTGCCCCCAGCGTGTTCATCTTCCCTC |
| | | CTAGCGACGAGCAGCTGAAGAGCGGCACCGCTAGCGTGGTGTGT |
| | | CTGCTGAATAACTTCTATCCCAGGGAGGCCAAGGTGCAGTGGAA |
| | | GGTGGATAACGCCCTGCAGAGCGGCAACTCCCAGGAGTCCGTGA |
| | | CCGAGCAGGACTCCAAGGACAGCACCTACTCCCTGAGCTCCACCC |
| | | TGACCCTGTCCAAGGCTGATTATGAGAAGCACAAGGTGTATGCTT |
| | | GCGAGGTGACACACCAGGGCCTGTCCAGCCCTGTGACCAAGAGC |
| | | TTCAACCGGGGCGAGTGC |
| A133- | 71 | AACATCCAGTTGACCCAGTCTCCATCCTCCCTGTCTGCATCTGTAG |
| LC | | GAGACAGAGTCACCATCACTTGCCGGGCTAGTCAGGGCATTGAA |
| | | ACTGATTTGGCCTGGTATCAGCAAAAACCAGGGAAAGCCCCTAA |
| | | GCGCCTGATTTATGGTGCCTCCACTTCACAAAACGGGGTTCCGTC |
| | | AAGGTTCAGTGGCGGTGGATATGGGACACACTTCACTCTCACAAT |
| | | CAGCGGCCTACAGCCTGAGGACTTTGCAACTTATTACTGTTTACA |
| | | GCATAACAACTTTCCCTACACTTTTGGCCAGGGGACCAGGCTGGA |
| | | GATCAAGAGGACCGTGGCTGCCCCCAGCGTGTTCATCTTCCCTCC |
| | | TAGCGACGAGCAGCTGAAGAGCGGCACCGCTAGCGTGGTGTGTC |
| | | TGCTGAATAACTTCTATCCCAGGGAGGCCAAGGTGCAGTGGAAG |
| | | GTGGATAACGCCCTGCAGAGCGGCAACTCCCAGGAGTCCGTGAC |
| | | CGAGCAGGACTCCAAGGACAGCACCTACTCCCTGAGCTCCACCCT |
| | | GACCCTGTCCAAGGCTGATTATGAGAAGCACAAGGTGTATGCTTG |
| | | CGAGGTGACACACCAGGGCCTGTCCAGCCCTGTGACCAAGAGCT |
| | | TCAACCGGGGCGAGTGC |
| A154- | 72 | GAAATTGTGATGACACAGTCTCCAGCCACCCTGTCTTTGTCTCCA |
| LC | | GGGGAAAGAGCCACCCTCTCCTGCAGGGCCAGTCAGAGTGTTAG |
| | | CAGCTACTTAGCCTGGTACCAACAGAAACCTGGCCAGGCTCCCAG |
| | | GCTCCTCATCTATGATGCATCCAACAGGGCCACTGGCATCCCAGC |
| | | CAGGTTCAGTGGCAGTGGGTCTGGGACAGACTTCACTCTCACCAT |
| | | CAGCAGCCTAGAGCCTGAAGATTTTGCAGTTTATTACTGTCAGCA |
| | | GCGTAGCAACTGGCCGCTCACTTTCGGCGGAGGGACCAAGGTGG |
| | | AGATCAAAAGGACCGTGGCTGCCCCCAGCGTGTTCATCTTCCCTC |
| | | CTAGCGACGAGCAGCTGAAGAGCGGCACCGCTAGCGTGGTGTGT |
| | | CTGCTGAATAACTTCTATCCCAGGGAGGCCAAGGTGCAGTGGAA |
| | | GGTGGATAACGCCCTGCAGAGCGGCAACTCCCAGGAGTCCGTGA |
| | | CCGAGCAGGACTCCAAGGACAGCACCTACTCCCTGAGCTCCACCC |
| | | TGACCCTGTCCAAGGCTGATTATGAGAAGCACAAGGTGTATGCTT |
| | | GCGAGGTGACACACCAGGGCCTGTCCAGCCCTGTGACCAAGAGC |
| | | TTCAACCGGGGCGAGTGC |

Heavy Chains

| | | |
|---|---|---|
| A57- | 73 | CAGGTCCAGCTTGTGCAATCTGGGGCTGAGGTGAAGAAGCCTGG |
| HC | | GTCCTCGGTGAAGGTCTCCTGCAAGGCTTCTGGAGGCACCTTCAG |
| | | CAGCTATGCTATCAGCTGGGTGCGACAGGCCCCTGGACAAGGGC |
| | | TTGAGTGGATGGGAAGGATCATCCCTATCCTTGGTATAGCAAACT |
| | | ACGCACAGAAGTTCCAGGGCAGAGTCACGATTACCGCGGACAAA |
| | | TCCACGAGCACAGCCTACATGGAGCTGAGCAGCCTGAGATCTGA |
| | | GGACACGGCCGTGTATTACTGTGCGAGAGTAGTACCAGCTTACTA |
| | | CTACGGTATGGACGTCTGGGGCCAAGGGACCACGGTCACCGTCTC |
| | | ATCAGCTTCCACCAAGGGCCCCTCCGTGTTCCCCCTGGCTCCCTCT |
| | | TCCAAGAGCACCAGCGGCGGCACCGCTGCTCTGGGATGTCTGGTG |
| | | AAGGACTACTTCCCTGAGCCTGTGACCGTGTCCTGGAATTCCGGC |
| | | GCCCTGACCTCCGGCGTGCACACATTCCCTGCTGTGCTGCAGTCC |
| | | TCCGGCCTGTATAGCCTGTCCTCCGTGGTGACAGTGCCTAGCTCC |
| | | AGCCTGGGCACCCAGACCTATATCTGCAACGTGAACCACAAGCCT |
| | | AGCAATACCAAGGTGGACAAGAAGGTGGAGCCTAAGAGCTGCGA |
| | | CAAGACCCACACCTGTCCTCCATGTCCTGCTCCAGAACTGCTCGG |
| | | CGGACCTTCCGTGTTCCTGTTTCCTCCAAAGCCTAAGGACACCCT |
| | | GATGATCAGCAGAACCCCTGAAGTGACCTGCGTGGTGGTGGATG |
| | | TGTCCCACGAGGATCCCGAAGTGAAGTTCAATTGGTACGTGGACG |
| | | GCGTGGAAGTGCACAACGCCAAGACCAAGCCTAGAGAGGAACAG |
| | | TACAACAGCACCTACAGAGTGGTGTCCGTGCTGACCGTGCTGCAC |
| | | CAGGATTGGCTGAACGGCAAAGAGTACAAGTGCAAGGTGTCCAA |
| | | CAAGGCCCTGCCTGCTCCTATCGAGAAAACCATCAGCAAGGCCA |
| | | AGGGCCAGCCTAGGGAACCCCAGGTTTACACACTGCCTCCAAGC |
| | | AGGGACGAGCTGACCAAGAATCAGGTGTCCCTGACCTGCCTGGT |
| | | CAAGGGCTTCTACCCTTCCGATATCGCCGTGGAATGGGAGAGCAA |
| | | TGGCCAGCCTGAGAACAACTACAAGACAACCCCTCCTGTGCTGG |
| | | ACAGCGACGGCTCATTCTTCCTGTACAGCAAGCTGACAGTGGACA |
| | | AGAGCAGATGGCAGCAGGGCAACGTGTTCAGCTGCAGCGTGATG |
| | | CACGAGGCCCTGCACAACCACTACACCCAGAAGTCCCTGAGCCT |
| | | GTCTCCTGGCAAA |
| A96- | 74 | CAGGTTCAGCTGGTACAGTCTGGGGCTGAGGTGAAGAAGCCTGG |
| HC | | GTCCTCGGTGAAGGTCTCCTGCAAGGCTTCTGGAGGCACCTTCAG |
| | | CAGCTATGCTATCAGCTGGGTGCGACAGGCCCCTGGACAAGGGC |
| | | TTGAGTGGATGGGAGGGATCATCCCTATCTTTGGTACAGCAAACT |
| | | ACGCACAGAAGTTCCAGGGCAGAGTCACGATTACCGCGGACGAA |
| | | TCCACGAGCACAGCCTACATGGAGCTGAGCAGCCTGAGATCTGA |
| | | GGACACGGCCGTGTATTACTGTGCGAGAGTAAGGAGGGGTTATT |
| | | ATGCTTTTGATATCTGGGGCCAAGGGACAATGGTCACCGTCTCAT |
| | | CAGCTTCCACCAAGGGCCCCTCCGTGTTCCCCCTGGCTCCCTCTTC |
| | | CAAGAGCACCAGCGGCGGCACCGCTGCTCTGGGATGTCTGGTGA |
| | | AGGACTACTTCCCTGAGCCTGTGACCGTGTCCTGGAATTCCGGCG |
| | | CCCTGACCTCCGGCGTGCACACATTCCCTGCTGTGCTGCAGTCCT |
| | | CCGGCCTGTATAGCCTGTCCTCCGTGGTGACAGTGCCTAGCTCCA |
| | | GCCTGGGCACCCAGACCTATATCTGCAACGTGAACCACAAGCCTA |
| | | GCAATACCAAGGTGGACAAGAAGGTGGAGCCTAAGAGCTGCGAC |
| | | AAGACCCACACCTGTCCTCCATGTCCTGCTCCAGAACTGCTCGGC |
| | | GGACCTTCCGTGTTCCTGTTTCCTCCAAAGCCTAAGGACACCCTG |
| | | ATGATCAGCAGAACCCCTGAAGTGACCTGCGTGGTGGTGGATGT |
| | | GTCCCACGAGGATCCCGAAGTGAAGTTCAATTGGTACGTGGACG |
| | | GCGTGGAAGTGCACAACGCCAAGACCAAGCCTAGAGAGGAACAG |
| | | TACAACAGCACCTACAGAGTGGTGTCCGTGCTGACCGTGCTGCAC |
| | | CAGGATTGGCTGAACGGCAAAGAGTACAAGTGCAAGGTGTCCAA |
| | | CAAGGCCCTGCCTGCTCCTATCGAGAAAACCATCAGCAAGGCCA |
| | | AGGGCCAGCCTAGGGAACCCCAGGTTTACACACTGCCTCCAAGC |
| | | AGGGACGAGCTGACCAAGAATCAGGTGTCCCTGACCTGCCTGGT |
| | | CAAGGGCTTCTACCCTTCCGATATCGCCGTGGAATGGGAGAGCAA |
| | | TGGCCAGCCTGAGAACAACTACAAGACAACCCCTCCTGTGCTGG |
| | | ACAGCGACGGCTCATTCTTCCTGTACAGCAAGCTGACAGTGGACA |
| | | AGAGCAGATGGCAGCAGGGCAACGTGTTCAGCTGCAGCGTGATG |
| | | CACGAGGCCCTGCACAACCACTACACCCAGAAGTCCCTGAGCCT |
| | | GTCTCCTGGCAAA |
| A115- | 75 | CAGGTACAGCTGCAGCAGTCTGGGGCTGAGGTGAAGAAGCCTGG |
| HC | | GTCCTCGGTGAAGGTCTCCTGCAAGGCTTCTGGAGGCACCTTCAG |
| | | CAGCTATGCTATCAGCTGGGTGCGACAGGCCCCTGGACAAGGGC |
| | | TTGAGTGGATGGGAAGGATCATCCCTATCCTTGGTATAGCAAACT |
| | | ACGCACAGAAGTTCCAGGGCAGAGTCACGATTACCGCGGACAAA |
| | | TCCACGAGCACAGCCTACATGGAGCTGAGCAGCCTGAGATCTGA |
| | | GGACACGGCCGTGTATTACTGTGCGAGAGAGCCAAGTAGTAGTG |
| | | GTTATCCTTCCGGTGACTACTGGGGCCAGGGAACCCTGGTCACCG |
| | | TCTCATCAGCTTCCACCAAGGGCCCCTCCGTGTTCCCCCTGGCTCC |
| | | CTCTTCCAAGAGCACCAGCGGCGGCACCGCTGCTCTGGGATGTCT |
| | | GGTGAAGGACTACTTCCCTGAGCCTGTGACCGTGTCCTGGAATTC |
| | | CGGCGCCCTGACCTCCGGCGTGCACACATTCCCTGCTGTGCTGCA |
| | | GTCCTCCGGCCTGTATAGCCTGTCCTCCGTGGTGACAGTGCCTAG |
| | | CTCCAGCCTGGGCACCCAGACCTATATCTGCAACGTGAACCACAA |
| | | GCCTAGCAATACCAAGGTGGACAAGAAGGTGGAGCCTAAGAGCT |
| | | GCGACAAGACCCACACCTGTCCTCCATGTCCTGCTCCAGAACTGC |
| | | TCGGCGGACCTTCCGTGTTCCTGTTTCCTCCAAAGCCTAAGGACA |
| | | CCCTGATGATCAGCAGAACCCCTGAAGTGACCTGCGTGGTGGTGG |
| | | ATGTGTCCCACGAGGATCCCGAAGTGAAGTTCAATTGGTACGTGG |
| | | ACGGCGTGGAAGTGCACAACGCCAAGACCAAGCCTAGAGAGGAA |
| | | CAGTACAACAGCACCTACAGAGTGGTGTCCGTGCTGACCGTGCTG |
| | | CACCAGGATTGGCTGAACGGCAAAGAGTACAAGTGCAAGGTGTC |
| | | CAACAAGGCCCTGCCTGCTCCTATCGAGAAAACCATCAGCAAGG |
| | | CCAAGGGCCAGCCTAGGGAACCCCAGGTTTACACACTGCCTCCA |
| | | AGCAGGGACGAGCTGACCAAGAATCAGGTGTCCCTGACCTGCCT |
| | | GGTCAAGGGCTTCTACCCTTCCGATATCGCCGTGGAATGGGAGAG |
| | | CAATGGCCAGCCTGAGAACAACTACAAGACAACCCCTCCTGTGCT |
| | | GGACAGCGACGGCTCATTCTTCCTGTACAGCAAGCTGACAGTGGA |
| | | CAAGAGCAGATGGCAGCAGGGCAACGTGTTCAGCTGCAGCGTGA |
| | | TGCACGAGGCCCTGCACAACCACTACACCCAGAAGTCCCTGAGC |
| | | CTGTCTCCTGGCAAA |
| A117- | 76 | CAGGTGCAGCTGGTACAGTCTGGGGCTGAGGTGAAGAAGCCTGG |
| HC | | GTCCTCGGTGAAGGTCTCCTGCAAGGCTTCTGGAGGCACCTTCAG |
| | | CAGCTATGCTATCAGCTGGGTGCGACAGGCCCCTGGACAAGGGC |
| | | TTGAGTGGATGGGAAGGATCATCCCTATCCTTGGTATAGCAAACT |
| | | ACGCACAGAAGTTCCAGGGCAGAGTCACGATTACCGCGGACAAA |
| | | TCCACGAGCACAGCCTACATGGAGCTGAGCAGCCTGAGATCTGA |
| | | GGACACGGCCGTGTATTACTGTGCGAGAATGAGGGGTGATACTG |
| | | CTAACTTTGACTACTGGGGCCAGGGAACCCTGGTCACCGTCTCAT |
| | | CAGCTTCCACCAAGGGCCCCTCCGTGTTCCCCCTGGCTCCCTCTTC |
| | | CAAGAGCACCAGCGGCGGCACCGCTGCTCTGGGATGTCTGGTGA |
| | | AGGACTACTTCCCTGAGCCTGTGACCGTGTCCTGGAATTCCGGCG |
| | | CCCTGACCTCCGGCGTGCACACATTCCCTGCTGTGCTGCAGTCCT |
| | | CCGGCCTGTATAGCCTGTCCTCCGTGGTGACAGTGCCTAGCTCCA |
| | | GCCTGGGCACCCAGACCTATATCTGCAACGTGAACCACAAGCCTA |
| | | GCAATACCAAGGTGGACAAGAAGGTGGAGCCTAAGAGCTGCGAC |
| | | AAGACCCACACCTGTCCTCCATGTCCTGCTCCAGAACTGCTCGGC |
| | | GGACCTTCCGTGTTCCTGTTTCCTCCAAAGCCTAAGGACACCCTG |
| | | ATGATCAGCAGAACCCCTGAAGTGACCTGCGTGGTGGTGGATGT |
| | | GTCCCACGAGGATCCCGAAGTGAAGTTCAATTGGTACGTGGACG |
| | | GCGTGGAAGTGCACAACGCCAAGACCAAGCCTAGAGAGGAACAG |
| | | TACAACAGCACCTACAGAGTGGTGTCCGTGCTGACCGTGCTGCAC |
| | | CAGGATTGGCTGAACGGCAAAGAGTACAAGTGCAAGGTGTCCAA |
| | | CAAGGCCCTGCCTGCTCCTATCGAGAAAACCATCAGCAAGGCCA |
| | | AGGGCCAGCCTAGGGAACCCCAGGTTTACACACTGCCTCCAAGC |
| | | AGGGACGAGCTGACCAAGAATCAGGTGTCCCTGACCTGCCTGGT |
| | | CAAGGGCTTCTACCCTTCCGATATCGCCGTGGAATGGGAGAGCAA |
| | | TGGCCAGCCTGAGAACAACTACAAGACAACCCCTCCTGTGCTGG |
| | | ACAGCGACGGCTCATTCTTCCTGTACAGCAAGCTGACAGTGGACA |
| | | AGAGCAGATGGCAGCAGGGCAACGTGTTCAGCTGCAGCGTGATG |
| | | CACGAGGCCCTGCACAACCACTACACCCAGAAGTCCCTGAGCCT |
| | | GTCTCCTGGCAAA |
| A125- | 77 | CAGGTTCAGCTGGTACAGTCTGGGGCTGAGGTGAAGAAGCCTGG |
| HC | | GTCCTCGGTGAAGGTCTCCTGCAAGGCTTCTGGAGGCACCTTCAG |
| | | CAGCTATGCTATCAGCTGGGTGCGACAGGCCCCTGGACAAGGGC |
| | | TTGAGTGGATGGGAAGGATCATCCCTATCCTTGGTATAGCAAACT |
| | | ACGCACAGAAGTTCCAGGGCAGAGTCACGATTACCGCGGACAAA |
| | | TCCACGAGCACAGCCTACATGGAGCTGAGCAGCCTGAGATCTGA |
| | | GGACACGGCCGTGTATTACTGTGCGAGAGATTTCGGGGGTTATTA |
| | | CTCCGGCGCTTTTGATATCTGGGGCCAAGGGACAATGGTCACCGT |
| | | CTCATCAGCTTCCACCAAGGGCCCCTCCGTGTTCCCCCTGGCTCCC |
| | | TCTTCCAAGAGCACCAGCGGCGGCACCGCTGCTCTGGGATGTCTG |
| | | GTGAAGGACTACTTCCCTGAGCCTGTGACCGTGTCCTGGAATTCC |
| | | GGCGCCCTGACCTCCGGCGTGCACACATTCCCTGCTGTGCTGCAG |
| | | TCCTCCGGCCTGTATAGCCTGTCCTCCGTGGTGACAGTGCCTAGC |
| | | TCCAGCCTGGGCACCCAGACCTATATCTGCAACGTGAACCACAAG |
| | | CCTAGCAATACCAAGGTGGACAAGAAGGTGGAGCCTAAGAGCTG |
| | | CGACAAGACCCACACCTGTCCTCCATGTCCTGCTCCAGAACTGCT |
| | | CGGCGGACCTTCCGTGTTCCTGTTTCCTCCAAAGCCTAAGGACAC |
| | | CCTGATGATCAGCAGAACCCCTGAAGTGACCTGCGTGGTGGTGG |
| | | ATGTGTCCCACGAGGATCCCGAAGTGAAGTTCAATTGGTACGTGG |
| | | ACGGCGTGGAAGTGCACAACGCCAAGACCAAGCCTAGAGAGGAA |
| | | CAGTACAACAGCACCTACAGAGTGGTGTCCGTGCTGACCGTGCTG |
| | | CACCAGGATTGGCTGAACGGCAAAGAGTACAAGTGCAAGGTGTC |
| | | CAACAAGGCCCTGCCTGCTCCTATCGAGAAAACCATCAGCAAGG |
| | | CCAAGGGCCAGCCTAGGGAACCCCAGGTTTACACACTGCCTCCA |
| | | AGCAGGGACGAGCTGACCAAGAATCAGGTGTCCCTGACCTGCCT |
| | | GGTCAAGGGCTTCTACCCTTCCGATATCGCCGTGGAATGGGAGAG |
| | | CAATGGCCAGCCTGAGAACAACTACAAGACAACCCCTCCTGTGCT |
| | | GGACAGCGACGGCTCATTCTTCCTGTACAGCAAGCTGACAGTGGA |
| | | CAAGAGCAGATGGCAGCAGGGCAACGTGTTCAGCTGCAGCGTGA |
| | | TGCACGAGGCCCTGCACAACCACTACACCCAGAAGTCCCTGAGC |
| | | CTGTCTCCTGGCAAA |
| A131- | 78 | CAGGTACAGCTGCAGCAGTCTGGGGCTGAGGTGAAGAAGCCTGG |
| HC | | GTCCTCGGTGAAGGTCTCCTGCAAGGCTTCTGGAGGCACCTTCAG |
| | | CAGCTATGCTATCAGCTGGGTGCGACAGGCCCCTGGACAAGGGC |
| | | TTGAGTGGATGGGAAGGATCATCCCTATCCTTGGTATAGCAAACT |
| | | ACGCACAGAAGTTCCAGGGCAGAGTCACGATTACCGCGGACAAA |
| | | TCCACGAGCACAGCCTACATGGAGCTGAGCAGCCTGAGATCTGA |
| | | GGACACGGCCGTGTATTACTGTGCGAGAGAGCCAAGTAGTAGTG |
| | | GTTATCCTTCCGGTGACTACTGGGGCCAGGGAACCCTGGTCACCG |
| | | TCTCATCAGCTTCCACCAAGGGCCCCTCCGTGTTCCCCCTGGCTCC |
| | | CTCTTCCAAGAGCACCAGCGGCGGCACCGCTGCTCTGGGATGTCT |
| | | GGTGAAGGACTACTTCCCTGAGCCTGTGACCGTGTCCTGGAATTC |
| | | CGGCGCCCTGACCTCCGGCGTGCACACATTCCCTGCTGTGCTGCA |
| | | GTCCTCCGGCCTGTATAGCCTGTCCTCCGTGGTGACAGTGCCTAG |
| | | CTCCAGCCTGGGCACCCAGACCTATATCTGCAACGTGAACCACAA |
| | | GCCTAGCAATACCAAGGTGGACAAGAAGGTGGAGCCTAAGAGCT |
| | | GCGACAAGACCCACACCTGTCCTCCATGTCCTGCTCCAGAACTGC |
| | | TCGGCGGACCTTCCGTGTTCCTGTTTCCTCCAAAGCCTAAGGACA |
| | | CCCTGATGATCAGCAGAACCCCTGAAGTGACCTGCGTGGTGGTGG |
| | | ATGTGTCCCACGAGGATCCCGAAGTGAAGTTCAATTGGTACGTGG |
| | | ACGGCGTGGAAGTGCACAACGCCAAGACCAAGCCTAGAGAGGAA |
| | | CAGTACAACAGCACCTACAGAGTGGTGTCCGTGCTGACCGTGCTG |
| | | CACCAGGATTGGCTGAACGGCAAAGAGTACAAGTGCAAGGTGTC |
| | | CAACAAGGCCCTGCCTGCTCCTATCGAGAAAACCATCAGCAAGG |
| | | CCAAGGGCCAGCCTAGGGAACCCCAGGTTTACACACTGCCTCCA |
| | | AGCAGGGACGAGCTGACCAAGAATCAGGTGTCCCTGACCTGCCT |
| | | GGTCAAGGGCTTCTACCCTTCCGATATCGCCGTGGAATGGGAGAG |
| | | CAATGGCCAGCCTGAGAACAACTACAAGACAACCCCTCCTGTGCT |
| | | GGACAGCGACGGCTCATTCTTCCTGTACAGCAAGCTGACAGTGGA |
| | | CAAGAGCAGATGGCAGCAGGGCAACGTGTTCAGCTGCAGCGTGA |
| | | TGCACGAGGCCCTGCACAACCACTACACCCAGAAGTCCCTGAGC |
| | | CTGTCTCCTGGCAAA |
| A133- | 79 | GAGGTGCAGCTGGTGCAGTCTGGGGCTGAGGTGAAGAAGCCTGG |
| HC | | GTCCTCGGTGAAGGTCTCCTGCAAGGCTTCTGGAGGCACCTTCAG |
| | | CAGCTATGCTATCAGCTGGGTGCGACAGGCCCCTGGACAAGGGC |
| | | TTGAGTGGATGGGAAGGATCATCCCTATCCTTGGTATAGCAAACT |
| | | ACGCACAGAAGTTCCAGGGCAGAGTCACGATTACCGCGGACAAA |
| | | TCCACGAGCACAGCCTACATGGAGCTGAGCAGCCTGAGATCTGA |
| | | GGACACGGCCGTGTATTACTGTGCGAGAGATCAGAGGCGCGGGG |
| | | GTAAGGTCGGTATGGACGTCTGGGGCCAAGGGACCCTGGTCACC |
| | | GTCTCATCAGCTTCCACCAAGGGCCCCTCCGTGTTCCCCCTGGCTC |
| | | CCTCTTCCAAGAGCACCAGCGGCGGCACCGCTGCTCTGGGATGTC |
| | | TGGTGAAGGACTACTTCCCTGAGCCTGTGACCGTGTCCTGGAATT |
| | | CCGGCGCCCTGACCTCCGGCGTGCACACATTCCCTGCTGTGCTGC |
| | | AGTCCTCCGGCCTGTATAGCCTGTCCTCCGTGGTGACAGTGCCTA |
| | | GCTCCAGCCTGGGCACCCAGACCTATATCTGCAACGTGAACCACA |
| | | AGCCTAGCAATACCAAGGTGGACAAGAAGGTGGAGCCTAAGAGC |
| | | TGCGACAAGACCCACACCTGTCCTCCATGTCCTGCTCCAGAACTG |
| | | CTCGGCGGACCTTCCGTGTTCCTGTTTCCTCCAAAGCCTAAGGAC |
| | | ACCCTGATGATCAGCAGAACCCCTGAAGTGACCTGCGTGGTGGTG |
| | | GATGTGTCCCACGAGGATCCCGAAGTGAAGTTCAATTGGTACGTG |
| | | GACGGCGTGGAAGTGCACAACGCCAAGACCAAGCCTAGAGAGGA |
| | | ACAGTACAACAGCACCTACAGAGTGGTGTCCGTGCTGACCGTGCT |
| | | GCACCAGGATTGGCTGAACGGCAAAGAGTACAAGTGCAAGGTGT |
| | | CCAACAAGGCCCTGCCTGCTCCTATCGAGAAAACCATCAGCAAG |
| | | GCCAAGGGCCAGCCTAGGGAACCCCAGGTTTACACACTGCCTCC |
| | | AAGCAGGGACGAGCTGACCAAGAATCAGGTGTCCCTGACCTGCC |
| | | TGGTCAAGGGCTTCTACCCTTCCGATATCGCCGTGGAATGGGAGA |
| | | GCAATGGCCAGCCTGAGAACAACTACAAGACAACCCCTCCTGTG |
| | | CTGGACAGCGACGGCTCATTCTTCCTGTACAGCAAGCTGACAGTG |
| | | GACAAGAGCAGATGGCAGCAGGGCAACGTGTTCAGCTGCAGCGT |
| | | GATGCACGAGGCCCTGCACAACCACTACACCCAGAAGTCCCTGA |
| | | GCCTGTCTCCTGGCAAA |
| A154- | 80 | CAGGTCCAGCTGGTACAGTCTGGAGCAGAGGTGAAAAAGCCTGG |
| HC | | GTCCTCGGTGAAGGTCTCCTGCAAGGCTTCTGGAGGCACCTTCAG |
| | | CAGCTATGCTATCAGCTGGGTGCGACAGGCCCCTGGACAAGGGC |
| | | TTGAGTGGATGGGAAGGATCATCCCTATCCTTGGTATAGCAAACT |
| | | ACGCACAGAAGTTCCAGGGCAGAGTCACGATTACCGCGGACAAA |
| | | TCCACGAGCACAGCCTACATGGAGCTGAGCAGCCTGAGATCTGA |
| | | GGACACGGCCGTGTATTACTGTGCGAGAGAACCCCCATACAGTG |
| | | GGAGCTACTCGTACGGTATGGACGTCTGGGGCCAAGGGACAATG |
| | | GTCACCGTCTCATCAGCTTCCACCAAGGGCCCCTCCGTGTTCCCC |
| | | CTGGCTCCCTCTTCCAAGAGCACCAGCGGCGGCACCGCTGCTCTG |
| | | GGATGTCTGGTGAAGGACTACTTCCCTGAGCCTGTGACCGTGTCC |
| | | TGGAATTCCGGCGCCCTGACCTCCGGCGTGCACACATTCCCTGCT |
| | | GTGCTGCAGTCCTCCGGCCTGTATAGCCTGTCCTCCGTGGTGACA |
| | | GTGCCTAGCTCCAGCCTGGGCACCCAGACCTATATCTGCAACGTG |
| | | AACCACAAGCCTAGCAATACCAAGGTGGACAAGAAGGTGGAGCC |
| | | TAAGAGCTGCGACAAGACCCACACCTGTCCTCCATGTCCTGCTCC |
| | | AGAACTGCTCGGCGGACCTTCCGTGTTCCTGTTTCCTCCAAAGCC |
| | | TAAGGACACCCTGATGATCAGCAGAACCCCTGAAGTGACCTGCG |
| | | TGGTGGTGGATGTGTCCCACGAGGATCCCGAAGTGAAGTTCAATT |
| | | GGTACGTGGACGGCGTGGAAGTGCACAACGCCAAGACCAAGCCT |
| | | AGAGAGGAACAGTACAACAGCACCTACAGAGTGGTGTCCGTGCT |
| | | GACCGTGCTGCACCAGGATTGGCTGAACGGCAAAGAGTACAAGT |
| | | GCAAGGTGTCCAACAAGGCCCTGCCTGCTCCTATCGAGAAAACC |
| | | ATCAGCAAGGCCAAGGGCCAGCCTAGGGAACCCCAGGTTTACAC |
| | | ACTGCCTCCAAGCAGGGACGAGCTGACCAAGAATCAGGTGTCCC |
| | | TGACCTGCCTGGTCAAGGGCTTCTACCCTTCCGATATCGCCGTGG |
| | | AATGGGAGAGCAATGGCCAGCCTGAGAACAACTACAAGACAACC |
| | | CCTCCTGTGCTGGACAGCGACGGCTCATTCTTCCTGTACAGCAAG |
| | | CTGACAGTGGACAAGAGCAGATGGCAGCAGGGCAACGTGTTCAG |
| | | CTGCAGCGTGATGCACGAGGCCCTGCACAACCACTACACCCAGA |
| | | AGTCCCTGAGCCTGTCTCCTGGCAAA |

In one embodiment the invention provides a human anti-PD-L2 antibody comprising a light chain consisting of variable regions LCDR1, LCDR2, and LCDR3, and a heavy chain consisting of consisting of variable regions HCDR1, HCDR2, and HCDR3.

In one embodiment, the invention provides LCDR1 of Seq. ID 7 or at least an amino acid sequence which is 89% identical to Seq. ID 7. LCDR1 is a nine amino acid sequence long polypeptide. Said LCDR1 consists of amino acid Asn (N) or Tyr (Y) at position 6. Furthermore, LCDR1 is of Seq. ID 43. LCDR2 is of Seq. ID 8 or at least an amino acid sequence which is 85% or more identical to Seq. ID 8. LCDR2 comprises a thirteen amino acid long sequence with at least 85% identity to Seq. ID 8 consisting of amino acid Gly (G) or Asp (D) at position 7, and amino acid Thr (T) or Asn (N) at position 10. Furthermore, LCDR2 is of Seq. ID 14. LCDR3 is of Seq. ID 9 or at least 50% or more identical to Seq. ID 9. LCDR3 comprises a fourteen amino acid long sequence with at least 50% or more identity to Seq. ID 9 consisting of amino acid His (H) or Gln (Q) at position 2, amino acid Gln (Q) or Arg (R) at position 3, amino acid Gly (G) or Asn (N) or Ser(S) at position 4, no amino acid or amino acid Asn (N) at position 5, no amino acid or amino acid Trp (W) at position 6, no amino acid or amino acid Pro (P) at position 7, no amino acid or amino acid Ile (I) or Leu (L) at position 8, amino acid Gln (Q) or Gly (G) at position 12. Furthermore, LCDR3 is selected from the group consisting of Seq. ID 15 and 45.

In yet another embodiment, the invention provides HCDR1 of Seq. ID 10. HCDR2 is of Seq. ID 11 or at least 81% or more identical to Seq. ID 11. HCDR2 comprises a sixteen amino acid long sequence with at least 81% identity to Seq. ID 11 consisting of amino acid Gly (G) or Arg (R) at position 6, amino acid Phe (F) or Leu (L) at position 11, and amino acid Thr (T) or Ile (I) at position 13. Furthermore, HCDR2 is of Seq. ID 17. HCDR3 is of Seq. ID 12. Furthermore, HCDR3 is selected from the group consisting of Seq. ID. 18 and 48.

In yet another embodiment, the human anti-PD-L2 antibody comprises of light chain consisting of variable region LCDR1 of Seq. ID 7, LCDR2 of Seq. ID 8, and LCDR3 of Seq. ID 9, and the heavy chain consists of variable region HCDR1 of Seq. ID 10, HCDR2 of Seq. ID 11, and HCDR3 of Seq. ID 12.

In yet another embodiment, the human anti-PD-L2 antibody comprises of light chain consisting of variable region LCDR1 of Seq. ID 13 which is 100% identical to Seq. ID 7, LCDR2 of Seq. ID 14, and LCDR3 of Seq. ID 15, and the heavy chain consists of variable region HCDR1 of Seq. ID 16 which is 100% identical to Seq. ID 10, HCDR2 of Seq. ID 17, and HCDR3 of Seq. ID 18.

In yet another embodiment, the human anti-PD-L2 antibody comprises of light chain consisting of variable region LCDR1 of Seq. ID 43, LCDR2 of Seq. ID 44 which is 100% identical to Seq. ID 14, and LCDR3 of Seq. ID 45, and the heavy chain consists of variable region HCDR1 of Seq. ID 46 which is 100% identical to Seq. ID 10, HCDR2 of Seq. ID 17 which is 100% identical to Seq. ID 17, and HCDR3 of Seq. ID 48.

In yet another embodiment, a human anti-PD-L2 antibody consisting of a light chain of Seq. ID 50 or at least 92% or more identical to Seq. ID 50, and a heavy chain consisting of Seq. ID 58 or at least at least 97% or more identical to Seq. ID 58. Said light chain is further selected from the group consisting of Seq. ID 51 and 56. Said heavy chain is further selected from the group consisting of Seq. ID 59 and 64.

The invention further provides, a human anti-PD-L2 antibody consisting of a light chain of Seq. ID 51 and a heavy chain of Seq. ID 59.

The invention also provides, a human anti-PD-L2 antibody consisting of a light chain of Seq. ID 56 and a heavy chain of Seq. ID 64.

Said light chain of Seq. ID 50 consists of variable region LCDR1 of Seq. ID 7, LCDR2 of Seq. ID 8, and LCDR3 of Seq. ID 9. Furthermore, said heavy chain of Seq. ID 58 consists of variable region HCDR1 of Seq. ID 10, HCDR2 of Seq. ID 11, and HCDR3 of Seq. ID 12.

Said light chain of Seq. ID 51 consists of variable region LCDR1 of Seq. ID 7, LCDR2 of Seq. ID 14, and LCDR3 of Seq. ID 15. Furthermore, said heavy chain of Seq. ID 59 consists of variable region HCDR1 of Seq. ID 10, HCDR2 of Seq. ID 17, and HCDR3 of Seq. ID 18.

Said light chain of Seq. ID 56 consists of variable region LCDR1 of Seq. ID 43, LCDR2 of Seq. ID 14, and LCDR3 of Seq. ID 45. Furthermore, said heavy chain of Seq. ID 64 consists of variable region HCDR1 of Seq. ID 10, HCDR2 of Seq. ID 17, and HCDR3 of Seq. ID 48.

In yet another embodiment, the human anti-PD-L2 antibody comprises light chain of Seq. ID 51, and heavy chain of Seq. ID 59.

In yet another embodiment, the human anti-PD-L2 antibody comprises light chain of Seq. ID 56, and heavy chain of Seq. ID 64.

In yet another embodiment, the invention provides a nucleotide sequence of Seq. ID 66 encoding light chain of human anti-PD-L2 antibody of Seq. ID 50.

In yet another embodiment, the invention provides a nucleotide sequence of Seq. ID 74 encoding heavy chain of human anti-PD-L2 antibody of Seq. ID 58.

In yet another embodiment, the invention provides a nucleotide sequence of Seq. ID 67 encoding light chain of human anti-PD-L2 antibody of Seq. ID 51.

In yet another embodiment, the invention provides a nucleotide sequence of Seq. ID 75 encoding heavy chain of human anti-PD-L2 antibody of Seq. ID 59.

In yet another embodiment, the invention provides a nucleotide sequence of Seq. ID 72 encoding light chain of human anti-PD-L2 antibody of Seq. ID 56.

In yet another embodiment, the invention provides a nucleotide sequence of Seq. ID 80 encoding heavy chain of human anti-PD-L2 antibody of Seq. ID 64.

In an alternate embodiment, the invention provides a human anti-PD-L2 antibody, wherein, light chain consists of variable region LCDR1 of Seq. ID 31, LCDR2 of Seq. ID 32, and LCDR3 of Seq. ID 33, and heavy chain consists of variable region HCDR1 of Seq. ID 34 which is 100% identical to Seq. ID 10, HCDR2 of Seq. ID 35, and HCDR3 of Seq. ID 36.

In another embodiment, the human anti-PD-L2 antibody consists of a light chain of Seq. ID 54, and a heavy chain of Seq. ID 62.

Said light chain of Seq. ID 54 consists of variable region LCDR1 of Seq. ID 31, LCDR2 of Seq. ID 32, and LCDR3 of Seq. ID 33. Furthermore, said heavy chain of Seq. ID 62 consists of variable region HCDR1 of Seq. ID 10, HCDR2 of Seq. ID 35, and HCDR3 of Seq. ID 36.

In another embodiment, the invention provides a nucleotide sequence of Seq. ID 70 encoding light chain of human anti-PD-L2 antibody of Seq. ID 54, and a nucleotide sequence of Seq. ID 78 encoding heavy chain of human anti-PD-L2 antibody of Seq. ID 62.

In yet another alternate embodiment, the invention provides a LCDR1 of Seq. ID 25 or at least 65% or more identical to Seq. ID 25. Furthermore, LCDR1 is of Seq. ID 37. LCDR2 of Seq. ID 26 or at least 78% or more identical to Seq. ID 26. The LCDR2 comprises a thirteen amino acid long sequence with at least 78% or more identity to Seq. ID 26 consisting of amino acid Lys (L) or Arg (R) at position 3, and amino acid Ala (A) or Glu (G) at position 7. Furthermore, LCDR2 is of Seq. ID 38. LCDR3 of Seq. ID 27 at least 69% or more identical to Seq. ID 27. Furthermore, LCDR3 is of Seq. ID 39.

In yet another alternate embodiment, the invention provides a HCDR1 of Seq. ID 28 and Seq. ID 40 which are 100% identical to Seq. ID 10. The invention also provides HCDR2 of Seq. ID 29 and Seq. ID 41 which are 100% identical to Seq. ID 17. HCDR3 is selected from the group consisting of Seq. ID 30 and Seq. ID 42.

In yet another embodiment, the human anti-PD-L2 antibody comprises of light chain consisting of variable region LCDR1 of Seq. ID 25, LCDR2 of Seq. ID 26, and LCDR3 of Seq. ID 27, and the heavy chain consists of variable region HCDR1 of Seq. ID 10, HCDR2 of Seq. ID 17, and HCDR3 of Seq. ID 30.

In yet another embodiment, the human anti-PD-L2 antibody comprises of light chain consisting of variable region LCDR1 of Seq. ID 37, LCDR2 of Seq. ID 38, and LCDR3 of Seq. ID 39, and the heavy chain consists of variable region HCDR1 of Seq. ID 10, HCDR2 of Seq. ID 17, and HCDR3 of Seq. ID 42.

In yet another embodiment, a human anti-PD-L2 antibody consisting of a light chain of Seq. ID 53 or at least 89% or more identical to Seq. ID 53, and a heavy chain consisting of Seq. ID 61 or at least at least 97% or more identical to Seq. ID 61. Said light chain of Seq. ID 55. Said heavy chain is of Seq. ID 63.

The invention also provides, a human anti-PD-L2 antibody consisting of a light chain of Seq. ID 55 and a heavy chain of Seq. ID 63.

Said light chain of Seq. ID 53 consists of variable region LCDR1 of Seq. ID 25, LCDR2 of Seq. ID 26, and LCDR3 of Seq. ID 27. Furthermore, said heavy chain of Seq. ID 61 consists of variable region HCDR1 of Seq. ID 10, HCDR2 of Seq. ID 17, and HCDR3 of Seq. ID 30.

Said light chain of Seq. ID 55 consists of variable region LCDR1 of Seq. ID 37, LCDR2 of Seq. ID 38, and LCDR3 of Seq. ID 39. Furthermore, said heavy chain of Seq. ID 63 consists of variable region HCDR1 of Seq. ID 10, HCDR2 of Seq. ID 17, and HCDR3 of Seq. ID 42.

In yet another embodiment, a human anti-PD-L2 antibody consisting of a light chain of Seq. ID 55, and a heavy chain consisting of Seq. ID 63.

In yet another embodiment, the invention provides a nucleotide sequence of Seq. ID 69 encoding light chain of human anti-PD-L2 antibody of Seq. ID 53.

In yet another embodiment, the invention provides a nucleotide sequence of Seq. ID 77 encoding heavy chain of human anti-PD-L2 antibody of Seq. ID 61.

In yet another embodiment, the invention provides a nucleotide sequence of Seq. ID 71 encoding light chain of human anti-PD-L2 antibody of Seq. ID 55.

In yet another embodiment, the invention provides a nucleotide sequence of Seq. ID 79 encoding heavy chain of human anti-PD-L2 antibody of Seq. ID 63.

In yet another alternate embodiment, the invention provides a LCDR1 of Seq. ID 1 or at least 73% or more identical to Seq. ID 1. Furthermore, LCDR1 is of Seq. ID 19. LCDR2 of Seq. ID 2 or at least 85% or more identical to Seq. ID 2. Furthermore, LCDR2 is of Seq. ID 20. LCDR3 is selected from the group consisting of Seq. ID 3 and Seq. ID 21.

In yet another alternate embodiment, the invention provides a HCDR1 of Seq. ID 4 and Seq. ID 22 which are 100% identical to Seq. ID 10. HCDR2 is selected from the group consisting of Seq. ID 5 and Seq. ID 23 which are identical Seq. ID 17. HCDR3 is selected from the group consisting of Seq. ID 6 and Seq. ID 24.

In yet another embodiment, the human anti-PD-L2 antibody comprises of light chain consisting of variable region LCDR1 of Seq. ID 1, LCDR2 of Seq. ID 2, and LCDR3 of Seq. ID 3, and the heavy chain consists of variable region HCDR1 of Seq. ID 10, HCDR2 of Seq. ID 17, and HCDR3 of Seq. ID 6.

In yet another embodiment, the human anti-PD-L2 antibody comprises of light chain consisting of variable region LCDR1 of Seq. ID 19, LCDR2 of Seq. ID 20, and LCDR3 of Seq. ID 21, and the heavy chain consists of variable region HCDR1 of Seq. ID 10, HCDR2 of Seq. ID 17, and HCDR3 of Seq. ID 24.

In yet another embodiment, a human anti-PD-L2 antibody consisting of a light chain of Seq. ID 49, and a heavy chain consisting of Seq. ID 57.

The invention also provides, a human anti-PD-L2 antibody consisting of a light chain of Seq. ID 52 and a heavy chain of Seq. ID 60.

Said light chain of Seq. ID 49 consists of variable region LCDR1 of Seq. ID 1, LCDR2 of Seq. ID 2, and LCDR3 of Seq. ID 3. Furthermore, said heavy chain of Seq. ID 57 consists of variable region HCDR1 of Seq. ID 10, HCDR2 of Seq. ID 17, and HCDR3 of Seq. ID 6.

Said light chain of Seq. ID 52 consists of variable region LCDR1 of Seq. ID 19, LCDR2 of Seq. ID 20, and LCDR3 of Seq. ID 21. Furthermore, said heavy chain of Seq. ID 60 consists of variable region HCDR1 of Seq. ID 10, HCDR2 of Seq. ID 17, and HCDR3 of Seq. ID 24.

In yet another embodiment, the invention provides a nucleotide sequence of Seq. ID 65 encoding light chain of human anti-PD-L2 antibody of Seq. ID 49.

In yet another embodiment, the invention provides a nucleotide sequence of Seq. ID 73 encoding heavy chain of human anti-PD-L2 antibody of Seq. ID 57.

In yet another embodiment, the invention provides a nucleotide sequence of Seq. ID 68 encoding light chain of human anti-PD-L2 antibody of Seq. ID 52.

In yet another embodiment, the invention provides a nucleotide sequence of Seq. ID 76 encoding heavy chain of human anti-PD-L2 antibody of Seq. ID 60.

In yet another embodiment, the present invention provides a method for production of recombinant monoclonal human anti-PD-L2 antibodies. The antibody may be produced by techniques known in the art. The nucleotide sequences encoding the light chains and heavy chains of antibodies are cloned in suitable vectors for expression in host cells. The method comprises the steps of:
a. Transforming suitable host cells with expression vectors containing nucleotide sequences encoding light and heavy chains of antibodies;
b. Isolating/separating/collecting antibodies produced by the host cells; and
c. Purification of antibodies.

In yet another embodiment, the present invention provides kits comprising of human anti-PD-L2 antibodies or their antibody fragments thereof useful for detection of presence of PD-L2 in a biological sample. The antibody may further be conjugated with labelling molecules. Further, the antibody may be attached to a substrate or a device for use in assays such as ELISA etc.

In yet another embodiment, the present invention provides a pharmaceutical composition comprising of human anti-PD-L2 antibodies or their antibody fragments thereof and one or more pharmaceutically acceptable carriers. The pharmaceutical compositions can be liquid solutions, suspensions, emulsions, capsules, tablets, sustained release formulation, and powder. Further, these pharmaceutical compositions may for administration as parenteral or non-parenteral routes.

The following examples are provided to better illustrate the claimed invention and are not to be interpreted as limiting the scope of the invention. All specific compositions, materials, and methods described below, in whole or in part, fall within the scope of the present invention. These specific compositions, materials, and methods are not intended to limit the invention, but merely to illustrate specific embodiments falling within the scope of the invention. One skilled in the art may develop equivalent compositions, materials, and methods without the exercise of inventive capacity and without departing from the scope of the invention. It will be understood that many variations can be made in the procedures herein described while still remaining within the bounds of the present invention. It is the intention of the inventors that such variations are included within the scope of the invention.

### EXAMPLES

### Example 1

### Panning, and Screening of Recombinant Humanized Antibody Library

a. Kingfisher method

Background deduction was done by diluting and blocking the preserved natural library phage suspension with 2.5% BSA, incubating with Dynabeads, and collecting the phage incubated after negative screening. Dynabeads blocking and coating by binding and cleaning the magnetic beads according to the method of Kingfisher magnetic bead screening system, and incubating the magnetic beads with 2.5% BSA. Positive screening was done by incubating the phage suspension collected after negative screening with the Dynabeads which were coated with biotin labelled antigen and blocked, then binding and cleaning according to the method of the Kingfisher magnetic bead screening system. Phages were eluted with trypsin. Library plate was infected after elution, the phage solution was fully mixed with logarithmic SS320 cells, and then incubated at 37° C. for 30 minutes, and then Spread on 2YT-Car+-Tet+ plate, and incubating the culture overnight in 37° C. The eluted phage solution was diluted with 10 times gradient of logarithmic SS320 cells, incubated at 37° C. for 30 minutes, mixed well and dropped 2 μL on the plate and incubated culture overnight in 37° C. Calculating the input and output of phage, preparing the input phage by scraping the phage on the plate and conducting four rounds of screening. Each round of screening used 3 gradients of antigen concentration to screen and to obtain antibody clones with high affinity.

b. Immunotube Method

For background deduction the preserved natural library was blocked with 5% PBSM. Immunotubes were coated and blocked with diluted antigen overnight at 4° C., then cleaned it according to the method of immunotube screening system, and block it with 5% PBSM. Positive screening was done by incubating the phage suspension with the protein coated and blocked immunotube, and binding and cleaning it according to the method of immunotube screening system. Elution, Infection, titration were performed as explained in kingfisher method.
c. Panning pools were detected for positivity rate using ELISA.
d. Preliminary screening of monoclonal by ELISA: The antigen Human PD-L2-Q9BQ51-His was used to perform Phage ELISA preliminary screening on the monoclonal antibodies collected in the second and third rounds of panning, and the Human PD-L2-Q9BQ51-His coating method was used for screening. The negative control was 6G/6H, the corresponding output stock solution was 6A/6B, the positive control was 12G/12H. The positive clones were delineated according to a certain background value. 447 positive clones were obtained that bind to the antigen Human PD-L2-Q9BQ51-His through preliminary screening, of which 69 were molecules with unique sequences.
e. Affinity ranking using ELISA: 69 clones with unique sequences were diluted with gradient in the form of preparation Phage supernatant, and were subjected to affinity ELISA determination with the corresponding antigen Human PD-L2-Q9BQ51-His. The results showed that 54 clones with unique sequences had affinity with the antigen Human PD-L2-Q9BQ51-His.
f. Final clone selection: of 54 clones, 36 were chosen based on the affinity.

### Example 2

### Construction and Production of IgGs

DNA of the 36 best cell binding candidates were synthesized and inserted into pcDNA3.4 to make expression plasmids of full-length IgGs, fully human IgGs. The heavy and light chain expression plasmids were used to co-transfect Expi293F cells. The recombinant IgGs secreted to the medium were purified using protein A affinity chromatography.

### Example 3

### Analysis of Human IgGs

### 1. Assessment of Binding Activity of Selected Monoclonal Antibody Clones

The binding activity of selected 36 monoclonal antibody clones was assessed by FACS using HuPD-L2-HEK293 which are HEK293 cells overexpressing human PD-L2 (HuPD-L2). The binding activity was tested against the well-known and reference PD-L2 antibody-24F.10C12.

TABLE 3
Sample layout for FACS Binding of candidate
antibodies on huPD L2 HEK293 cells.

| | | | | |
|---|---|---|---|---|
| A3(P16005) | A29(P16013) | A63(P16021) | A97(P16029) | A137(P16037) |
| A5(P16006) | A32(P16014) | A71(P16022) | A109(P16030) | A138(P16038) |
| A12(P16007) | A38(P16015) | A78(P16023) | A115(P16031) | A141(P16039) |
| A13(P16008) | A43(P16016) | A82(P16024) | A117(P16032) | A154(P16040) |
| A14(P16009) | A51(P16017) | A84(P16025) | A125(P16033) | 24F.10C12(P08047) |
| A19(P16010) | A55(P16018) | A89(P16026) | A128(P16034) | IgG1(P02183) |
| A20(P16011) | A57(P16019) | A94(P16027) | A131(P16035) | cell + sec |
| A22(P16012) | A58(P16020) | A96(P16028) | A133(P16036) | cell + only |

TABLE 4
FACS Binding assay results w.r.t. to the positive
control Ab 24F.10C12(P08047), IgG1 (negative
control) and other negative controls.

| | | | | |
|---|---|---|---|---|
| 575000 | 588000 | 652000 | 629000 | 439000 |
| 624000 | 442000 | 555000 | 621000 | 426000 |
| 664000 | 598000 | 628000 | 510000 | 665000 |
| 559000 | 645000 | 639000 | 485000 | 454000 |
| 478000 | 578000 | 595000 | 513000 | 707000 |
| 495000 | 603000 | 622000 | 68610 | 851 |
| 412000 | 506000 | 584000 | 491000 | 360 |
| 575000 | 588000 | 652000 | 629000 | 439000 |

All the 36 antibodies showed good binding to HuPD-L2 comparable to the positive reference antibody 24F.10C12.

### 2. Assessment of Blocking Activity of Selected Monoclonal Antibody Clones

The blocking activity of selected 36 monoclonal antibody clones was assessed by FACS using HuPD-L2-HEK293 cells and the ligand HumanPD-1-Q15116-Fc against the well-known and reference PD-L2 antibody-24F.10C12.

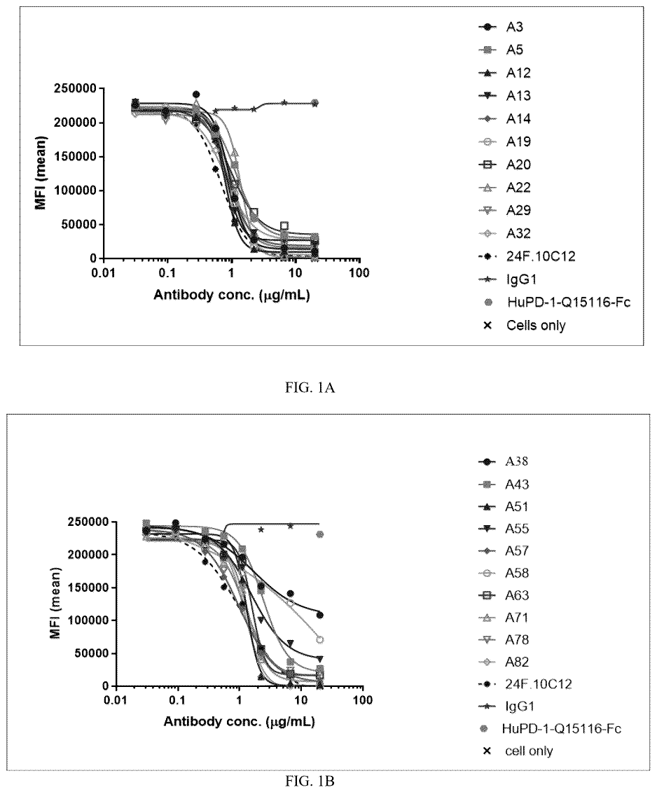

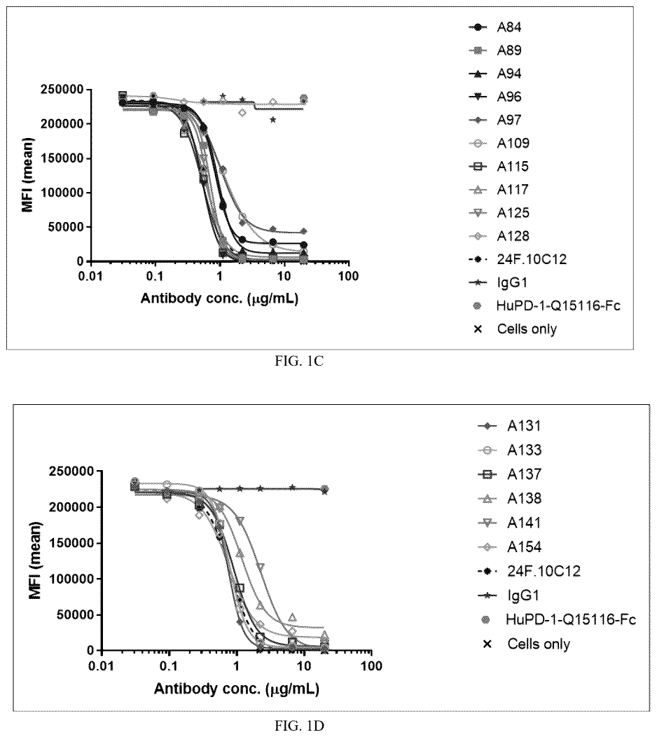

As shown in FIG. 1, of the 36 antibody clones only 1 did not show any blocking activity, and the remaining 35 showed good blocking activity against the ligand.

### 3. Selection of Final Anti-PD-L2 Antibody Clones for Further Assessment

Based on the binding and blocking activity efficiency out of the 35 clones, only 8 clones were selected for further analysis—namely A57, A96, A115, A117, A125, A131, A133 and A154. The amino acid sequences of light chain and heavy chain of the selected 8 candidates are provided in Table 1.

### 4. PD-1-NF-AT-Luciferase-Jurkat Reporter Assay

PD-1-NF-AT-luciferase-Jurkat Reporter Assay was conducted using CD3L PD L2 CHO cells with the positive control antibody 24F.10C12, IgG1 (negative control), and the candidate anti-PD-L2 antibodies—A57, A96, A115, A117, A125, A131, A133 and A154.

FIG. 2 shows that all the candidate antibodies showed blocking activity candidate molecules on the PD 1 NF AT luciferase Jurkat reporter system. However, the results show that the blocking effect of candidate molecules on PD 1 NF AT luciferase Jurkat reporter system was less than that of the positive antibody 24F.10C12.

### 5. EC50 Value of Candidate Antibodies

EC50 value is the concentration of antibody that gives half-maximal binding determined by direct and saturable binding of an antibody to both target antigen.

Table 5 provides the EC-50 values of the positive control antibody 24F.10C12, and the candidate anti-PD-L2 antibodies—A57, A96, A115, A117, A125, A131, A133 and A154.

TABLE 5
EC50 values of candidate antibodies

| Antibody | EC 50(nM) |
|---|---|
| A57 | 28.38 |
| A96 | 8.159 |
| A115 | 11.64 |
| A117 | 37.66 |
| A125 | 11.18 |
| A131 | 15.83 |
| A133 | 28.32 |
| A154 | 32.41 |
| 24F.10C12 | 5.25 |

The results show that the candidate antibodies show higher EC50 values than the positive control 24F.10C12 showing that higher concentrations of candidate antibodies are required to half maximal antigen binding than the positive control 24F.10C12.

Hence, of the selected candidate antibodies only three antibodies were finalized for carrying out the animal model in vivo studies to check the effect of the antibodies on tumour resistance, and tumour growth reduction. The selected antibodies were A96, A125, and A131.

### 6. In Vivo Efficacy Evaluation Using Animal Mouse Model

Cell line-derived xenograft (CDX) model refers to the subcutaneous or in situ tumour models established by subcutaneous, intravenous, or in situ inoculation of tumour cell lines into wild-type, immunodeficient, or humanized immune system mice. CDX models are featured by short experimental cycle, high success rate of modelling, and relatively low experimental cost, and are widely used as in vivo tumour functional models for drug target validation, pharmacodynamic evaluation, PK/PD studies, and multi-drug synergistic efficacy evaluation.

The objective of this study was to validate the tumour inhibitory effect of selected candidate antibodies-A96, A125, and A131 in comparison to reference antibody 24F.10C12.

Briefly, the tumour cell line was in vitro expanded appropriately and then inoculated into the right back of hPD-1 mice by subcutaneous injection to establish a tumour model. After the tumour grew to 50-80 mm³, the mice were randomized and administered with the reference antibody or candidate antibody according to the dose regimen. Then, the tumour size and signs were measured, and the corresponding efficacy was evaluated at 3-5 weeks after administration.

The tumour cell line used was PD-L2-MC38 cell which is a murine colon adenocarcinoma cells overexpressing PD-L2.

The resuspended PD-L2-MC38 cells were injected subcutaneously into the right back of mice at 0.1 mL per mouse. The specific grouping information is shown in Table 6.

TABLE 6
Grouping information for mice study on tumour
inhibition by candidate antibodies

| | | Number | | Dose | | |
|---|---|---|---|---|---|---|
| | | of | Dose | volume | | |
| Group | Name | animals | (mpk) | (mL/kg) | Route | Cycle |
| 1 | R15- | 6 | 2 | 10 | i.p. | BIW × 3 |
| | F7(IgG1) | | | | | |
| 2 | A96 | 6 | 2 | 10 | i.p. | BIW × 3 |
| 3 | A125 | 6 | 2 | 10 | i.p. | BIW × 3 |
| 4 | A131 | 6 | 2 | 10 | i.p. | BIW × 3 |
| 5 | 24F.10C12 | 6 | 2 | 10 | i.p. | BIW × 3 |
| 6 | 24F.10C12 | 3 | 5 | 10 | i.p. | BIW × 3 |
| 7 | A96 | 3 | 5 | 10 | i.p. | BIW × 3 |
| 8 | A125 | 3 | 5 | 10 | i.p. | BIW × 3 |
| 9 | A131 | 3 | 5 | 10 | i.p. | BIW × 3 |

Note:
Tumour volume = ½ (length diameter × width diameter × width diameter); i.p. stands for intraperitoneal injection; BIW stands for twice weekly.

After tumour inoculation, the effects of tumour growth and treatment on the normal behaviours of animals were tested, including the activity, food consumption and drinking, weight increase/decrease, eyes, coat, and other abnormalities of the laboratory animals. During the experiment, no abnormal behaviour or death was observed in the laboratory animals

### Results:

a) Weight: The weight of mice was measured twice weekly using an electronic balance.
b) Tumour measurement: After the laboratory mice were grouped, the tumour length and width diameters were measured twice weekly using a vernier calliper. When the tumour volume reached 2000 mm³, the tumour-bearing mice were euthanized with CO₂, and the tumour blocks were peeled off, weighed and photographed.

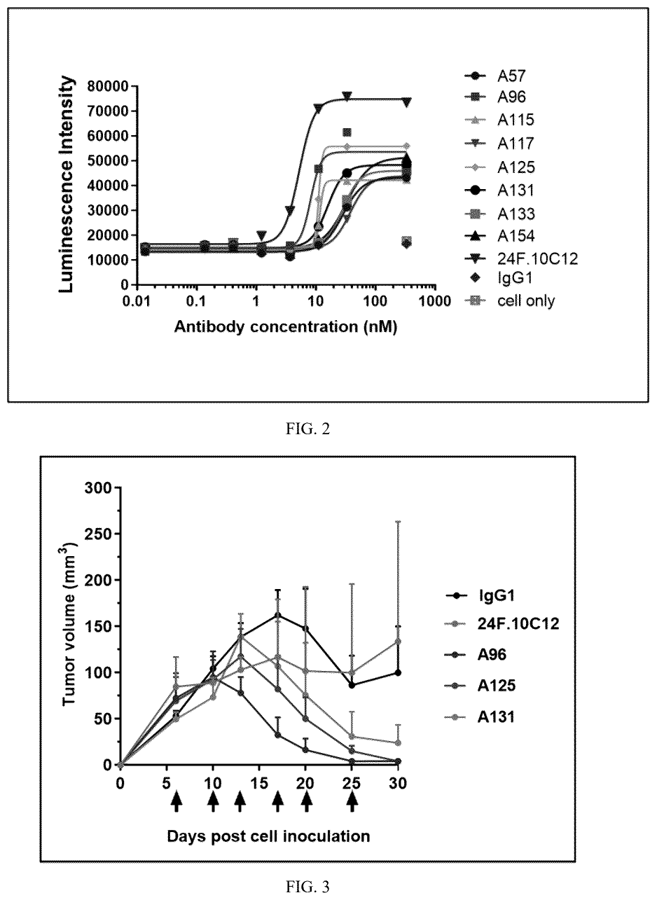

FIG. 3 provides a graphical representation of tumour growth upto 25 days post tumour cells inoculation and administration of either reference antibody or candidate antibody or IgG negative control at 2 doses 2 mpk or 5 mpk as listed in Table 6. Tumour growth was measured by measuring the length and width using vernier calliper. As shown, there was no effect of IgG negative control on the tumour growth and the tumour kept growing till day 16-17 and then starting slowly to reduce due other physiological effects. Reference antibody, A96, A125, and A131, showed reduction in tumour growth by day 10 and by day 25 the tumour was equal to that of negative control. This clearly showed that the reference antibody though was initially effective, later started showed reduced efficiency on tumour growth. At 2 mpk dose and more specifically at 5 mpk dose all the candidate antibodies-A96, A125, and A131 significantly showed reduction in tumour growth compared to reference antibody. At day 25, A96 antibody almost completely reduced the tumour.

This establishes that the candidate antibodies A96, A125, and A131 showed more efficacy compared to reference antibody A96, A125, and A131.

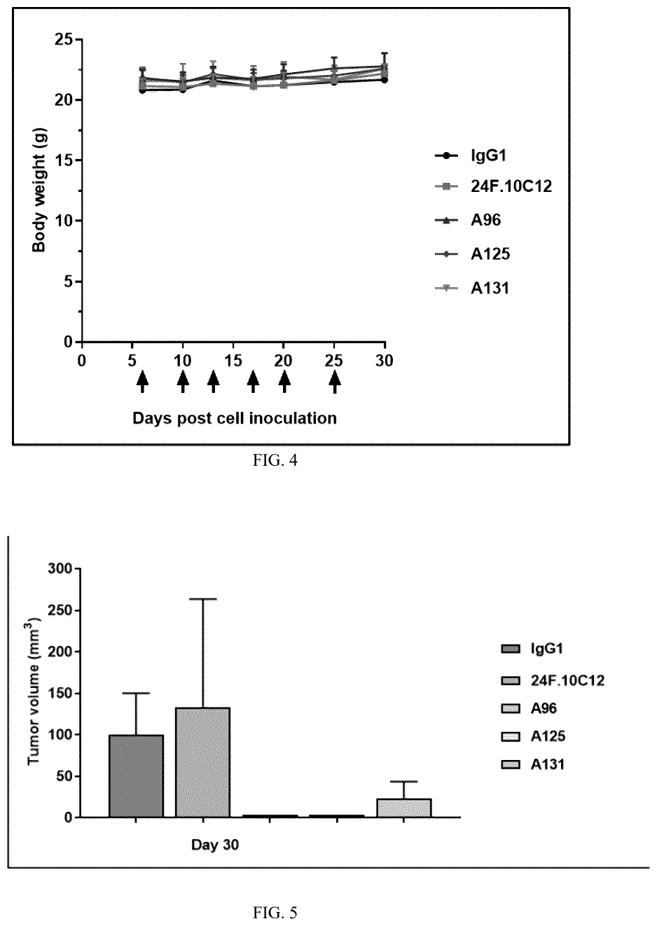

Further to show that the effect on tumour weight reduction was not related to overall mouse weight loss, the weight of all the mice was monitored throughout. FIG. 4 shows the mice weight over 25 days after tumour cell inoculation.

Further the efficacy of the antibodies on tumour inhibition was calculated using the method provided below.

### Calculation of Relative Tumour Inhibition Rate:

Method: TGI=1−T/C (%). T/C % refers to the relative tumour inhibition ratio, that is, the ratio of relative tumour volume or tumour weight between the treatment group and the control group at a certain time point; T and C refer to the relative tumour volume (RTV) or tumour weight (TW) of the treatment group and the control groups, respectively, at a specific time point.

For this purpose, R15-F7 (lgG1) (negative control), reference antibody or positive control (24F.10C12), A96, A125, and A131 at 5 mpk dose were used to complete the animal mouse study.

Table 7 provides the tumour inhibition ratio with the reference antibody 24F.10C12, candidate antibodies A96, A125, and A131 at 5 mpk dose at different time points compared to IgG1 negative control antibody.

TABLE 7
Tumour inhibition percentage/ratio

| | Dose | | | | | | |
|---|---|---|---|---|---|---|---|
| Group | (mpk) | D 10 | D 13 | D 17 | D 20 | D 25 | D 30 |
| R15-F7(IgG1) | 2 | 0.00 | 0.00 | 0.00 | 0.00 | 0.00 | 0.00 |
| 24F.10C12 | 5 | 14.84 | 25.71 | 28.12 | 31.06 | −15.81 | −33.93 |
| A96 | 5 | 9.05 | 43.83 | 79.99 | 88.98 | 95.37 | 95.99 |
| A125 | 5 | 11.29 | 15.39 | 49.49 | 66.15 | 82.67 | 95.99 |
| A131 | 5 | 29.50 | −0.17 | 34.13 | 48.75 | 64.35 | 76.19 |

The above results show that the negative control antibody had no effect on tumour growth, hence the inhibition rate is 0. The reference antibody, 24F.10C12, showed tumour inhibition till day 20, however, the efficacy drastically reduced after that which resulted in tumour regrowth more than the negative control antibody. The maximum effect 24F.10C12 could show on tumour weight was around 31% only by day 20.

Whereas A96 candidate antibody showed significant tumour inhibition and reached 80% inhibition by day 17, and by day 30 the tumour was reduced to 96% of the original weight.

A131 and A125 candidate antibodies also reduced tumour weight. A125 also showed 96% inhibition by Day 30, and A131 also showed significant tumour inhibition rate, around 76% by day 30, compared to reference antibody.

FIG. 5 provides a graphical representation of tumour weight at Day 30 using A96, A125, A131, 24F.10C12, and R15-F7 (lgG1) using 5 mpk dosage. The results clearly show that the tumour re-grows back when treated with reference antibody 24F.10C12. Whereas, A96, and A125 almost completely reduce the tumour and A131 significantly reduces the tumour growth compared to 24F.10C12.

These results clearly show that candidate antibodies A96, A125, and A131 are highly efficient over reference antibody—24F.10C12.

Table 8 summarises the light chain and heavy sequence IDs of candidate antibodies.

TABLE 8
Amino acid Sequence ID nos. of respective antibodies and the complementarity
determining regions (CDRs) of light chains and heavy chains

| | Sequence ID Nos. | | | | | | | |
|---|---|---|---|---|---|---|---|---|
| Antibody | Light | Heavy | | | | | | |
| name | Chain | Chain | LCDR1 | LCDR2 | LCDR3 | HCDR1 | HCDR2 | HCDR3 |
| A96 | 50 | 58 | 7 | 8 | 9 | 10 | 11 | 12 |
| A125 | 53 | 61 | 25 | 26 | 27 | 28 or 10 | 29 or 17 | 30 |
| A131 | 54 | 62 | 31 | 32 | 33 | 34 or 10 | 35 | 36 |

While certain exemplary embodiments have been described and shown in the accompanying drawings, it is to be understood that such embodiments are merely illustrative of, and not restrictive on, the broad invention, and that this invention not be limited to the specific constructions and arrangements shown and described, since various other changes, combinations, omissions, modifications and substitutions, in addition to those set forth in the above paragraphs, are possible. Those skilled in the art will appreciate that various adaptations and modifications of the just described embodiments can be configured without departing from the scope and spirit of the invention. Therefore, it is to be understood that, within the scope of the appended claims, the invention may be practiced other than as specifically described herein.

1. A human anti-PD-L2 antibody, wherein,
a light chain consists of variable region LCDR1 of SEQ ID No: 7, LCDR2 of SEQ ID No: 8, and LCDR3 of SEQ ID No: 9,
and the heavy chain consists of variable region HCDR1 of SEQ ID No: 10, HCDR2 of SEQ ID No: 11, and HCDR3 of SEQ ID No: 12.

2. The human anti-PD-L2 antibody as claimed in claim 1, wherein, the LCDR1 consists of SEQ ID No: 7.

3. (canceled)

4. The human anti-PD-L2 antibody as claimed in claim 1, wherein, the LCDR2 consists of SEQ ID No: 8.

5. (canceled)

6. The human anti-PD-L2 antibody as claimed in claim 1, wherein, the LCDR3 consists of SEQ ID No: 9.

7. (canceled)

8. The human anti-PD-L2 antibody as claimed in claim 1, wherein, the HCDR1 is of SEQ ID No: 10.

9. The human anti-PD-L2 antibody as claimed in claim 1, wherein, the HCDR2 consists of SEQ ID No: 11.

10. (canceled)

11. (canceled)

12. (canceled)

13. (canceled)

14. The human anti-PD-L2 antibody as claimed in claim 1, wherein, the light chain is of SEQ ID NO: 50, and the heavy chain is of SEQ ID NO: 58.

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. The human anti-PD-L2 antibody as claimed in claim 1, wherein, the HCDR3 is of SEQ ID NO: 12.

49. The human anti-PD-L2 antibody as claimed in claim 1, wherein the antibody is used in a pharmaceutical composition and a pharmaceutically acceptable carrier or excipient.

50. The human anti-PD-L2 antibody as claimed in claim 1, wherein, the antibody is used for treating solid tumors, liquid tumors and cancerous conditions either as a monotherapy or in combination therapy.

51. The human anti-PD-L2 antibody as claimed in claim 1, wherein, the antibody is used for treating infectious diseases.


SEQUENCE LISTING (80 sequences)

SEQ ID NO: 1 - protein, 11 aa, Homo sapiens
SSNVGTTYVSW

SEQ ID NO: 2 - protein, 13 aa, Homo sapiens
PKLLIYSNNQRPS

SEQ ID NO: 3 - protein, 16 aa, Homo sapiens
AAWDDSLNGRVFGGGT

SEQ ID NO: 4 - protein, 10 aa, Homo sapiens
SGGTFSSYAI

SEQ ID NO: 5 - protein, 16 aa, Homo sapiens
LEWMGRIIPILGIANY

SEQ ID NO: 6 - protein, 12 aa, Homo sapiens
VVPAYYYGMDVW

SEQ ID NO: 7 - protein, 9 aa, Homo sapiens
QSVSSNLAW

SEQ ID NO: 8 - protein, 13 aa, Homo sapiens
PRLLIYGASTRAT

SEQ ID NO: 9 - protein, 10 aa, Homo sapiens
QHQGTFGQGT

SEQ ID NO: 10 - protein, 10 aa, Homo sapiens
SGGTFSSYAI

SEQ ID NO: 11 - protein, 16 aa, Homo sapiens
LEWMGGIIPIFGTANY

SEQ ID NO: 12 - protein, 11 aa, Homo sapiens
VRRGYYAFDIW

SEQ ID NO: 13 - protein, 9 aa, Homo sapiens
QSVSSNLAW

SEQ ID NO: 14 - protein, 13 aa, Homo sapiens
PRLLIYDASNRAT

SEQ ID NO: 15 - protein, 14 aa, Homo sapiens
QQRNNWPITFGQGT

SEQ ID NO: 16 - protein, 10 aa, Homo sapiens
SGGTFSSYAI

SEQ ID NO: 17 - protein, 16 aa, Homo sapiens
LEWMGRIIPILGIANY

SEQ ID NO: 18 - protein, 13 aa, Homo sapiens
EPSSSGYPSGDYW

SEQ ID NO: 19 - protein, 11 aa, Homo sapiens
SSNIGNNYVSW

SEQ ID NO: 20 - protein, 13 aa, Homo sapiens
PKLLIYDNNKRPS

SEQ ID NO: 21 - protein, 16 aa, Homo sapiens
GTWDSSLSAWVFGGGT

SEQ ID NO: 22 - protein, 10 aa, Homo sapiens
SGGTFSSYAI

SEQ ID NO: 23 - protein, 16 aa, Homo sapiens
LEWMGRIIPILGIANY

SEQ ID NO: 24 - protein, 11 aa, Homo sapiens
MRGDTANFDYW

SEQ ID NO: 25 - protein, 9 aa, Homo sapiens
QGISSWLAW

SEQ ID NO: 26 - protein, 13 aa, Homo sapiens
PKLLIYAASSLQS

SEQ ID NO: 27 - protein, 14 aa, Homo sapiens
QQANSFPLTFGGGT

SEQ ID NO: 28 - protein, 10 aa, Homo sapiens
SGGTFSSYAI

SEQ ID NO: 29 - protein, 16 aa, Homo sapiens
LEWMGRIIPILGIANY

SEQ ID NO: 30 - protein, 13 aa, Homo sapiens
DFGGYYSGAFDIW

SEQ ID NO: 31 - protein, 9 aa, Homo sapiens
QGVDSSLNW

SEQ ID NO: 32 - protein, 13 aa, Homo sapiens
PRLLIYDASNRAT

SEQ ID NO: 33 - protein, 14 aa, Homo sapiens
QQRNNWPLTFGGGT

SEQ ID NO: 34 - protein, 10 aa, Homo sapiens
SGGTFSSYAI

SEQ ID NO: 35 - protein, 16 aa, Homo sapiens
LEWMGRIIPILGIANY

SEQ ID NO: 36 - protein, 13 aa, Homo sapiens
EPSSSGYPSGDYW

SEQ ID NO: 37 - protein, 9 aa, Homo sapiens
QGIETDLAW

SEQ ID NO: 38 - protein, 13 aa, Homo sapiens
PKRLIYGASTSQN

SEQ ID NO: 39 - protein, 14 aa, Homo sapiens
LQHNNFPYTFGQGT

SEQ ID NO: 40 - protein, 10 aa, Homo sapiens
SGGTFSSYAI

SEQ ID NO: 41 - protein, 16 aa, Homo sapiens
LEWMGRIIPILGIANY

SEQ ID NO: 42 - protein, 13 aa, Homo sapiens
DQRRGGKVGMDVW

SEQ ID NO: 43 - protein, 9 aa, Homo sapiens
QSVSSYLAW

SEQ ID NO: 44 - protein, 13 aa, Homo sapiens
PRLLIYDASNRAT

SEQ ID NO: 45 - protein, 14 aa, Homo sapiens
QQRSNWPLTFGGGT

SEQ ID NO: 46 - protein, 10 aa, Homo sapiens
SGGTFSSYAI

SEQ ID NO: 47 - protein, 16 aa, Homo sapiens
LEWMGRIIPILGIANY

SEQ ID NO: 48 - protein, 15 aa, Homo sapiens
EPPYSGSYSYGMDVW

SEQ ID NO: 49 - protein, 216 aa, Homo sapiens
QSVLTQPPSVSAAPGQTVTISCSGSSSNVGTTYVSWYQQLPGTAPKLLIYSNNQRPSGVPDRFSGSKSGTSASLAISGLQSEDEADYYCAAWDDSLNGRVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 50 - protein, 210 aa, Homo sapiens
EIVMTQSPATLSVSPGERATLSCRASQSVSSNLAWYQQKPGQAPRLLIYGASTRATGIPARFSGSGSGTEFTLTISSLQSEDFAVYYCQHQGTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 51 - protein, 214 aa, Homo sapiens
EIVLTQSPATLSLSPGERATLSCRASQSVSSNLAWYQQKPGQAPRLLIYDASNRATGIPARFSGSGSATDFTLTISSLEPEDFAVYYCQQRNNWPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 52 - protein, 216 aa, Homo sapiens
QSVLTQPPSVSAAPGQKVTISCSGSSSNIGNNYVSWYQQLPGTAPKLLIYDNNKRPSGIPDRFSGSKSGTSATLGITGLQTGDEADYYCGTWDSSLSAWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 53 - protein, 214 aa, Homo sapiens
DIQMIQSPSSVSASVGDRVTITCRASQGISSWLAWYQQKPGKAPKLLIYAASSLQSGVPSRFSGSGSGTDFTLTISSLQPEDFATYYCQQANSFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 54 - protein, 214 aa, Homo sapiens
EIVLTQSPAALSLSPGERATLSCRASQGVDSSLNWYQQKPGQAPRLLIYDASNRATGIPARFSGSGSGTDFTLTISSLEPEDFAVYYCQQRNNWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 55 - protein, 214 aa, Homo sapiens
NIQLTQSPSSLSASVGDRVTITCRASQGIETDLAWYQQKPGKAPKRLIYGASTSQNGVPSRFSGGGYGTHFTLTISGLQPEDFATYYCLQHNNFPYTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 56 - protein, 214 aa, Homo sapiens
EIVMTQSPATLSLSPGERATLSCRASQSVSSYLAWYQQKPGQAPRLLIYDASNRATGIPARFSGSGSGTDFTLTISSLEPEDFAVYYCQQRSNWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 57 - protein, 450 aa, Homo sapiens
QVQLVQSGAEVKKPGSSVKVSCKASGGTFSSYAISWVRQAPGQGLEWMGRIIPILGIANYAQKFQGRVTITADKSTSTAYMELSSLRSEDTAVYYCARVVPAYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 58 - protein, 449 aa, Homo sapiens
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARVRRGYYAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 59 - protein, 451 aa, Homo sapiens
QVQLQQSGAEVKKPGSSVKVSCKASGGTFSSYAISWVRQAPGQGLEWMGRIIPILGIANYAQKFQGRVTITADKSTSTAYMELSSLRSEDTAVYYCAREPSSSGYPSGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 60 - protein, 449 aa, Homo sapiens
QVQLVQSGAEVKKPGSSVKVSCKASGGTFSSYAISWVRQAPGQGLEWMGRIIPILGIANYAQKFQGRVTITADKSTSTAYMELSSLRSEDTAVYYCARMRGDTANFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 61 - protein, 451 aa, Homo sapiens
QVQLVQSGAEVKKPGSSVKVSCKASGGTFSSYAISWVRQAPGQGLEWMGRIIPILGIANYAQKFQGRVTITADKSTSTAYMELSSLRSEDTAVYYCARDFGGYYSGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 62 - protein, 451 aa, Homo sapiens; identical to SEQ ID NO: 59

SEQ ID NO: 63 - protein, 451 aa, Homo sapiens
EVQLVQSGAEVKKPGSSVKVSCKASGGTFSSYAISWVRQAPGQGLEWMGRIIPILGIANYAQKFQGRVTITADKSTSTAYMELSSLRSEDTAVYYCARDQRRGGKVGMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 64 - protein, 453 aa, Homo sapiens
QVQLVQSGAEVKKPGSSVKVSCKASGGTFSSYAISWVRQAPGQGLEWMGRIIPILGIANYAQKFQGRVTITADKSTSTAYMELSSLRSEDTAVYYCAREPPYSGSYSYGMDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 65 - DNA, 648 bp, Homo sapiens
  source 1..648: mol_type = other DNA
cagtctgtcttgacgcagccgccctcagtgtctgcggccccaggacagacggtcaccatctcctgctctggaagcagctccaacgttgggactacttatgtatcctggtaccagcagctcccaggaacggcccccaaactcctcatctatagtaataatcagcggccctcaggggtccctgaccgattctctggctccaagtctggcacctcagcctccctggccatcagtgggctccagtctgaggatgaggctgattattactgtgcagcatgggatgacagcctgaatggtcgagtgttcggcggagggaccaagctgaccgtcctaggacagcctaaggctgccccttccgtgacactgttccctccatcctctgaggaactgcaggccaacaaggctaccctcgtgtgcctgatctccgacttttaccctggcgctgtgaccgtggcctggaaggctgatagttctcctgtgaaggccggcgtggaaaccaccacaccttccaagcagtccaacaacaaatacgccgcctcctcctacctgtctctgacccctgaacagtggaagtcccaccggtcctacagctgccaagtgacccatgagggctccaccgtggaaaagaccgtggctcctaccgagtgctct

SEQ ID NO: 66 - DNA, 630 bp, Homo sapiens
  source 1..630: mol_type = other DNA
gaaattgtgatgacgcagtctccagccaccctgtctgtgtctccaggggaaagagccaccctctcctgcagggccagtcagagtgttagcagcaacttagcctggtaccagcagaaacctggccaggctcccaggctcctcatctatggtgcatccaccagggccactggtatcccagccaggttcagtggcagtgggtctgggacagagttcactctcaccatcagcagcctgcagtctgaagattttgcagtttattactgtcagcaccaggggacgttcggccaagggaccaaggtggagatcaaaaggaccgtggctgcccccagcgtgttcatcttccctcctagcgacgagcagctgaagagcggcaccgctagcgtggtgtgtctgctgaataacttctatcccagggaggccaaggtgcagtggaaggtggataacgccctgcagagcggcaactcccaggagtccgtgaccgagcaggactccaaggacagcacctactccctgagctccaccctgaccctgtccaaggctgattatgagaagcacaaggtgtatgcttgcgaggtgacacaccagggcctgtccagccctgtgaccaagagcttcaaccggggcgagtgc

SEQ ID NO: 67 - DNA, 642 bp, Homo sapiens
  source 1..642: mol_type = other DNA
gaaattgtgttgacacagtctccagccaccctgtctttgtctccaggggaaagagccaccctctcctgtagggccagtcagagtgttagcagcaacttagcctggtaccaacagaaacctggccaggctcccagactcctcatctatgatgcatccaacagggccactggcatcccagccaggttcagtggcagtgggtctgcgacagacttcactctcaccatcagcagcctagaacctgaagattttgcagtttattactgtcagcagcgtaacaactggccgatcaccttcggccaagggacacgactggagattaaaaggaccgtggctgcccccagcgtgttcatcttccctcctagcgacgagcagctgaagagcggcaccgctagcgtggtgtgtctgctgaataacttctatcccagggaggccaaggtgcagtggaaggtggataacgccctgcagagcggcaactcccaggagtccgtgaccgagcaggactccaaggacagcacctactccctgagctccaccctgaccctgtccaaggctgattatgagaagcacaaggtgtatgcttgcgaggtgacacaccagggcctgtccagccctgtgaccaagagcttcaaccggggcgagtgc

SEQ ID NO: 68 - DNA, 648 bp, Homo sapiens
  source 1..648: mol_type = other DNA
cagtctgtgctgacgcagccgccctcagtgtctgcggccccaggacagaaggtcaccatctcctgctctggaagcagctccaacattgggaataattatgtatcctggtaccagcagctcccaggaacagcccccaaactcctcatttatgacaataataagcgaccctcagggattcctgaccgattctctggctccaagtctggcacgtcagccaccctgggcatcaccggactccagactggggacgaggccgattattactgcggaacatgggatagcagcctgagtgcttgggtgttcggcggagggaccaagctgaccgtcctaggacagcctaaggctgccccttccgtgacactgttccctccatcctctgaggaactgcaggccaacaaggctaccctcgtgtgcctgatctccgacttttaccctggcgctgtgaccgtggcctggaaggctgatagttctcctgtgaaggccggcgtggaaaccaccacaccttccaagcagtccaacaacaaatacgccgcctcctcctacctgtctctgacccctgaacagtggaagtcccaccggtcctacagctgccaagtgacccatgagggctccaccgtggaaaagaccgtggctcctaccgagtgctct

SEQ ID NO: 69 - DNA, 642 bp, Homo sapiens
  source 1..642: mol_type = other DNA
gacatccagatgatccagtctccatcttccgtgtctgcatctgtaggagacagagtcaccatcacttgtcgggcgagtcagggtattagcagctggttagcctggtatcagcagaaaccagggaaagcccctaagctcctgatctatgctgcatccagtttgcaaagtggggtcccatcaaggttcagcggcagtggatctgggacagatttcactctcaccatcagcagcctgcagcctgaagattttgcaacttactattgtcaacaggctaacagtttccctctcactttcggcggagggaccaaggtggagatcaaaaggaccgtggctgcccccagcgtgttcatcttccctcctagcgacgagcagctgaagagcggcaccgctagcgtggtgtgtctgctgaataacttctatcccagggaggccaaggtgcagtggaaggtggataacgccctgcagagcggcaactcccaggagtccgtgaccgagcaggactccaaggacagcacctactccctgagctccaccctgaccctgtccaaggctgattatgagaagcacaaggtgtatgcttgcgaggtgacacaccagggcctgtccagccctgtgaccaagagcttcaaccggggcgagtgc

SEQ ID NO: 70 - DNA, 642 bp, Homo sapiens
  source 1..642: mol_type = other DNA
gaaattgtgttgacacagtctccagccgccctgtctttgtctccaggggaaagagccaccctctcctgcagggccagtcagggtgttgacagctccttaaactggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtaacaactggcctctcactttcggcggagggaccaaggtggagatcaaaaggaccgtggctgcccccagcgtgttcatcttccctcctagcgacgagcagctgaagagcggcaccgctagcgtggtgtgtctgctgaataacttctatcccagggaggccaaggtgcagtggaaggtggataacgccctgcagagcggcaactcccaggagtccgtgaccgagcaggactccaaggacagcacctactccctgagctccaccctgaccctgtccaaggctgattatgagaagcacaaggtgtatgcttgcgaggtgacacaccagggcctgtccagccctgtgaccaagagcttcaaccggggcgagtgc

SEQ ID NO: 71 - DNA, 642 bp, Homo sapiens
  source 1..642: mol_type = other DNA
aacatccagttgacccagtctccatcctccctgtctgcatctgtaggagacagagtcaccatcacttgccgggctagtcagggcattgaaactgatttggcctggtatcagcaaaaaccagggaaagcccctaagcgcctgatttatggtgcctccacttcacaaaacggggttccgtcaaggttcagtggcggtggatatgggacacacttcactctcacaatcagcggcctacagcctgaggactttgcaacttattactgtttacagcataacaactttccctacacttttggccaggggaccaggctggagatcaagaggaccgtggctgcccccagcgtgttcatcttccctcctagcgacgagcagctgaagagcggcaccgctagcgtggtgtgtctgctgaataacttctatcccagggaggccaaggtgcagtggaaggtggataacgccctgcagagcggcaactcccaggagtccgtgaccgagcaggactccaaggacagcacctactccctgagctccaccctgaccctgtccaaggctgattatgagaagcacaaggtgtatgcttgcgaggtgacacaccagggcctgtccagccctgtgaccaagagcttcaaccggggcgagtgc

SEQ ID NO: 72 - DNA, 642 bp, Homo sapiens
  source 1..642: mol_type = other DNA
gaaattgtgatgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggccgctcactttcggcggagggaccaaggtggagatcaaaaggaccgtggctgcccccagcgtgttcatcttccctcctagcgacgagcagctgaagagcggcaccgctagcgtggtgtgtctgctgaataacttctatcccagggaggccaaggtgcagtggaaggtggataacgccctgcagagcggcaactcccaggagtccgtgaccgagcaggactccaaggacagcacctactccctgagctccaccctgaccctgtccaaggctgattatgagaagcacaaggtgtatgcttgcgaggtgacacaccagggcctgtccagccctgtgaccaagagcttcaaccggggcgagtgc

SEQ ID NO: 73 - DNA, 1350 bp, Homo sapiens
  source 1..1350: mol_type = other DNA
caggtccagcttgtgcaatctggggctgaggtgaagaagcctgggtcctcggtgaaggtctcctgcaaggcttctggaggcaccttcagcagctatgctatcagctgggtgcgacaggcccctggacaagggcttgagtggatgggaaggatcatccctatccttggtatagcaaactacgcacagaagttccagggcagagtcacgattaccgcggacaaatccacgagcacagcctacatggagctgagcagcctgagatctgaggacacggccgtgtattactgtgcgagagtagtaccagcttactactacggtatggacgtctggggccaagggaccacggtcaccgtctcatcagcttccaccaagggcccctccgtgttccccctggctccctcttccaagagcaccagcggcggcaccgctgctctgggatgtctggtgaaggactacttccctgagcctgtgaccgtgtcctggaattccggcgccctgacctccggcgtgcacacattccctgctgtgctgcagtcctccggcctgtatagcctgtcctccgtggtgacagtgcctagctccagcctgggcacccagacctatatctgcaacgtgaaccacaagcctagcaataccaaggtggacaagaaggtggagcctaagagctgcgacaagacccacacctgtcctccatgtcctgctccagaactgctcggcggaccttccgtgttcctgtttcctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagcctagggaaccccaggtttacacactgcctccaagcagggacgagctgaccaagaatcaggtgtccctgacctgcctggtcaagggcttctacccttccgatatcgccgtggaatgggagagcaatggccagcctgagaacaactacaagacaacccctcctgtgctggacagcgacggctcattcttcctgtacagcaagctgacagtggacaagagcagatggcagcagggcaacgtgttcagctgcagcgtgatgcacgaggccctgcacaaccactacacccagaagtccctgagcctgtctcctggcaaa

SEQ ID NO: 74 - DNA, 1347 bp, Homo sapiens
  source 1..1347: mol_type = other DNA
caggttcagctggtacagtctggggctgaggtgaagaagcctgggtcctcggtgaaggtctcctgcaaggcttctggaggcaccttcagcagctatgctatcagctgggtgcgacaggcccctggacaagggcttgagtggatgggagggatcatccctatctttggtacagcaaactacgcacagaagttccagggcagagtcacgattaccgcggacgaatccacgagcacagcctacatggagctgagcagcctgagatctgaggacacggccgtgtattactgtgcgagagtaaggaggggttattatgcttttgatatctggggccaagggacaatggtcaccgtctcatcagcttccaccaagggcccctccgtgttccccctggctccctcttccaagagcaccagcggcggcaccgctgctctgggatgtctggtgaaggactacttccctgagcctgtgaccgtgtcctggaattccggcgccctgacctccggcgtgcacacattccctgctgtgctgcagtcctccggcctgtatagcctgtcctccgtggtgacagtgcctagctccagcctgggcacccagacctatatctgcaacgtgaaccacaagcctagcaataccaaggtggacaagaaggtggagcctaagagctgcgacaagacccacacctgtcctccatgtcctgctccagaactgctcggcggaccttccgtgttcctgtttcctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagcctagggaaccccaggtttacacactgcctccaagcagggacgagctgaccaagaatcaggtgtccctgacctgcctggtcaagggcttctacccttccgatatcgccgtggaatgggagagcaatggccagcctgagaacaactacaagacaacccctcctgtgctggacagcgacggctcattcttcctgtacagcaagctgacagtggacaagagcagatggcagcagggcaacgtgttcagctgcagcgtgatgcacgaggccctgcacaaccactacacccagaagtccctgagcctgtctcctggcaaa

SEQ ID NO: 75 - DNA, 1353 bp, Homo sapiens
  source 1..1353: mol_type = other DNA
caggtacagctgcagcagtctggggctgaggtgaagaagcctgggtcctcggtgaaggtctcctgcaaggcttctggaggcaccttcagcagctatgctatcagctgggtgcgacaggcccctggacaagggcttgagtggatgggaaggatcatccctatccttggtatagcaaactacgcacagaagttccagggcagagtcacgattaccgcggacaaatccacgagcacagcctacatggagctgagcagcctgagatctgaggacacggccgtgtattactgtgcgagagagccaagtagtagtggttatccttccggtgactactggggccagggaaccctggtcaccgtctcatcagcttccaccaagggcccctccgtgttccccctggctccctcttccaagagcaccagcggcggcaccgctgctctgggatgtctggtgaaggactacttccctgagcctgtgaccgtgtcctggaattccggcgccctgacctccggcgtgcacacattccctgctgtgctgcagtcctccggcctgtatagcctgtcctccgtggtgacagtgcctagctccagcctgggcacccagacctatatctgcaacgtgaaccacaagcctagcaataccaaggtggacaagaaggtggagcctaagagctgcgacaagacccacacctgtcctccatgtcctgctccagaactgctcggcggaccttccgtgttcctgtttcctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagcctagggaaccccaggtttacacactgcctccaagcagggacgagctgaccaagaatcaggtgtccctgacctgcctggtcaagggcttctacccttccgatatcgccgtggaatgggagagcaatggccagcctgagaacaactacaagacaacccctcctgtgctggacagcgacggctcattcttcctgtacagcaagctgacagtggacaagagcagatggcagcagggcaacgtgttcagctgcagcgtgatgcacgaggccctgcacaaccactacacccagaagtccctgagcctgtctcctggcaaa

SEQ ID NO: 76 - DNA, 1347 bp, Homo sapiens
  source 1..1347: mol_type = other DNA
caggtgcagctggtacagtctggggctgaggtgaagaagcctgggtcctcggtgaaggtctcctgcaaggcttctggaggcaccttcagcagctatgctatcagctgggtgcgacaggcccctggacaagggcttgagtggatgggaaggatcatccctatccttggtatagcaaactacgcacagaagttccagggcagagtcacgattaccgcggacaaatccacgagcacagcctacatggagctgagcagcctgagatctgaggacacggccgtgtattactgtgcgagaatgaggggtgatactgctaactttgactactggggccagggaaccctggtcaccgtctcatcagcttccaccaagggcccctccgtgttccccctggctccctcttccaagagcaccagcggcggcaccgctgctctgggatgtctggtgaaggactacttccctgagcctgtgaccgtgtcctggaattccggcgccctgacctccggcgtgcacacattccctgctgtgctgcagtcctccggcctgtatagcctgtcctccgtggtgacagtgcctagctccagcctgggcacccagacctatatctgcaacgtgaaccacaagcctagcaataccaaggtggacaagaaggtggagcctaagagctgcgacaagacccacacctgtcctccatgtcctgctccagaactgctcggcggaccttccgtgttcctgtttcctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagcctagggaaccccaggtttacacactgcctccaagcagggacgagctgaccaagaatcaggtgtccctgacctgcctggtcaagggcttctacccttccgatatcgccgtggaatgggagagcaatggccagcctgagaacaactacaagacaacccctcctgtgctggacagcgacggctcattcttcctgtacagcaagctgacagtggacaagagcagatggcagcagggcaacgtgttcagctgcagcgtgatgcacgaggccctgcacaaccactacacccagaagtccctgagcctgtctcctggcaaa

SEQ ID NO: 77 - DNA, 1353 bp, Homo sapiens
  source 1..1353: mol_type = other DNA
caggttcagctggtacagtctggggctgaggtgaagaagcctgggtcctcggtgaaggtctcctgcaaggcttctggaggcaccttcagcagctatgctatcagctgggtgcgacaggcccctggacaagggcttgagtggatgggaaggatcatccctatccttggtatagcaaactacgcacagaagttccagggcagagtcacgattaccgcggacaaatccacgagcacagcctacatggagctgagcagcctgagatctgaggacacggccgtgtattactgtgcgagagatttcgggggttattactccggcgcttttgatatctggggccaagggacaatggtcaccgtctcatcagcttccaccaagggcccctccgtgttccccctggctccctcttccaagagcaccagcggcggcaccgctgctctgggatgtctggtgaaggactacttccctgagcctgtgaccgtgtcctggaattccggcgccctgacctccggcgtgcacacattccctgctgtgctgcagtcctccggcctgtatagcctgtcctccgtggtgacagtgcctagctccagcctgggcacccagacctatatctgcaacgtgaaccacaagcctagcaataccaaggtggacaagaaggtggagcctaagagctgcgacaagacccacacctgtcctccatgtcctgctccagaactgctcggcggaccttccgtgttcctgtttcctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagcctagggaaccccaggtttacacactgcctccaagcagggacgagctgaccaagaatcaggtgtccctgacctgcctggtcaagggcttctacccttccgatatcgccgtggaatgggagagcaatggccagcctgagaacaactacaagacaacccctcctgtgctggacagcgacggctcattcttcctgtacagcaagctgacagtggacaagagcagatggcagcagggcaacgtgttcagctgcagcgtgatgcacgaggccctgcacaaccactacacccagaagtccctgagcctgtctcctggcaaa

SEQ ID NO: 78 - DNA, 1353 bp, Homo sapiens; identical to SEQ ID NO: 75
  source 1..1353: mol_type = other DNA

SEQ ID NO: 79 - DNA, 1353 bp, Homo sapiens
  source 1..1353: mol_type = other DNA
gaggtgcagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggaaggatcatccctatccttggtatagcaaactacgcacagaagttccagggcagagtcacgattaccgcggacaaatccacgagcacagcctacatggagctgagcagcctgagatctgaggacacggccgtgtattactgtgcgagagatcagaggcgcgggggtaaggtcggtatggacgtctggggccaagggaccctggtcaccgtctcatcagcttccaccaagggcccctccgtgttccccctggctccctcttccaagagcaccagcggcggcaccgctgctctgggatgtctggtgaaggactacttccctgagcctgtgaccgtgtcctggaattccggcgccctgacctccggcgtgcacacattccctgctgtgctgcagtcctccggcctgtatagcctgtcctccgtggtgacagtgcctagctccagcctgggcacccagacctatatctgcaacgtgaaccacaagcctagcaataccaaggtggacaagaaggtggagcctaagagctgcgacaagacccacacctgtcctccatgtcctgctccagaactgctcggcggaccttccgtgttcctgtttcctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagcctagggaaccccaggtttacacactgcctccaagcagggacgagctgaccaagaatcaggtgtccctgacctgcctggtcaagggcttctacccttccgatatcgccgtggaatgggagagcaatggccagcctgagaacaactacaagacaacccctcctgtgctggacagcgacggctcattcttcctgtacagcaagctgacagtggacaagagcagatggcagcagggcaacgtgttcagctgcagcgtgatgcacgaggccctgcacaaccactacacccagaagtccctgagcctgtctcctggcaaa

SEQ ID NO: 80 - DNA, 1359 bp, Homo sapiens
  source 1..1359: mol_type = other DNA
caggtccagctggtacagtctggagcagaggtgaaaaagcctgggtcctcggtgaaggtctcctgcaaggcttctggaggcaccttcagcagctatgctatcagctgggtgcgacaggcccctggacaagggcttgagtggatgggaaggatcatccctatccttggtatagcaaactacgcacagaagttccagggcagagtcacgattaccgcggacaaatccacgagcacagcctacatggagctgagcagcctgagatctgaggacacggccgtgtattactgtgcgagagaacccccatacagtgggagctactcgtacggtatggacgtctggggccaagggacaatggtcaccgtctcatcagcttccaccaagggcccctccgtgttccccctggctccctcttccaagagcaccagcggcggcaccgctgctctgggatgtctggtgaaggactacttccctgagcctgtgaccgtgtcctggaattccggcgccctgacctccggcgtgcacacattccctgctgtgctgcagtcctccggcctgtatagcctgtcctccgtggtgacagtgcctagctccagcctgggcacccagacctatatctgcaacgtgaaccacaagcctagcaataccaaggtggacaagaaggtggagcctaagagctgcgacaagacccacacctgtcctccatgtcctgctccagaactgctcggcggaccttccgtgttcctgtttcctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagcctagggaaccccaggtttacacactgcctccaagcagggacgagctgaccaagaatcaggtgtccctgacctgcctggtcaagggcttctacccttccgatatcgccgtggaatgggagagcaatggccagcctgagaacaactacaagacaacccctcctgtgctggacagcgacggctcattcttcctgtacagcaagctgacagtggacaagagcagatggcagcagggcaacgtgttcagctgcagcgtgatgcacgaggccctgcacaaccactacacccagaagtccctgagcctgtctcctggcaaa